(19) United States
(12) Patent Application Publication
(10) Pub. No.: US 2019/0225974 A1
(43) Pub. Date: Jul. 25, 2019

(54) TARGETED GENOME OPTIMIZATION IN PLANTS

(71) Applicant: BASF AGRICULTURAL SOLUTIONS SEED US LLC, Florham Park, NJ (US)

(72) Inventors: Katelijn D'HALLUIN, Zwijnaarde (BE); Eveline BOSSIER, Zwijnaarde (BE)

(21) Appl. No.: 16/336,045

(22) PCT Filed: Sep. 9, 2017
(86) PCT No.: PCT/EP2017/073658

§ 371 Date: Mar. 22, 2019

(30) Foreign Application Priority Data
Sep. 23, 2016 (EP) 16190292.9

(51) Int. Cl. C12N 15/82; C12N 9/22; C12N 15/90; C12N 15/11; C12N 15/62

(52) U.S. Cl. CPC C12N 15/8213; C12N 9/22; C12N 15/62; C12N 15/90; C12N 15/111; C12N 15/8205

(57) ABSTRACT

Improved methods and means are provided to modify in a targeted manner the genome of a plant cell at a predefined site using a nucleotide-guided DNA modifying polypeptide such as a RNA-guided endonuclease, a guide-polynucleotide and a donor molecule for repair of the DNA break.

### FIELD OF THE INVENTION

The invention relates to the field of agronomy. More particularly, the invention provides methods and means to introduce a targeted modification, including insertion, deletion or substitution, at a precisely localized nucleotide sequence in the genome of a plant cell. Specifically, the method employs an RNA-guided endonuclease (RGEN), a guide polynucleotide and a donor polynucleotide molecule which are delivered simultaneously to the plant cell via Agrobacterium-mediated delivery, resulting in an increase in the recovery of editing events wherein the donor polynucleotide has been used as a template for repair of a DNA break or one or more DNA nicks, or single strand DNA breaks, whether staggered or not. Also described is an assay for evaluating genome editing components.

### BACKGROUND

The need to introduce targeted modifications in genomes, such as plant genomes, including the control over the location of integration of foreign DNA has become increasingly important, and several methods have been developed in an effort to meet this need (for a review see Kumar and Fladung, 2001, Trends in Plant Science, 6, pp 155-159). These methods mostly rely on the initial introduction of a double stranded DNA break at the targeted location via expression of a double strand break inducing (DSBI) enzyme.

Activation of the target locus and/or repair or donor DNA through the induction of double stranded DNA breaks (DSB) via rare-cutting endonucleases, such as I-Scel has been shown to increase the frequency of homologous recombination by several orders of magnitude. (Puchta et al., 1996, Proc. Natl. Acad. Sci. U.S.A., 93, pp 5055-5060; Chilton and Que, Plant Physiol., 2003; D'Halluin et al. 2008 Plant Biotechnol. J. 6, 93-102).

WO 2005/049842 describes methods and means to improve targeted DNA insertion in plants using rare-cleaving “double stranded break” inducing (DSBI) enzymes, as well as improved I-Scel encoding nucleotide sequences.

WO2006/105946 describes a method for the exact exchange in plant cells and plants of a target DNA sequence for a DNA sequence of interest through homologous recombination, whereby the selectable or screenable marker used during the homologous recombination phase for temporal selection of the gene replacement events can subsequently be removed without leaving a foot-print and without resorting to in vitro culture during the removal step, employing the therein described method for the removal of a selected DNA by microspore specific expression of a DSBI rare-cleaving endonuclease.

WO2008/037436 describe variants of the methods and means of WO2006/105946 wherein the removal step of a selected DNA fragment induced by a double stranded break inducing rare cleaving endonuclease is under control of a germline-specific promoter. Other embodiments of the method relied on non-homologous end-joining at one end of the repair DNA and homologous recombination at the other end. WO08/148559 describes variants of the methods of WO2008/037436, i.e. methods for the exact exchange in eukaryotic cells, such as plant cells, of a target DNA sequence for a DNA sequence of interest through homologous recombination, whereby the selectable or screenable marker used during the homologous recombination phase for temporal selection of the gene replacement events can subsequently be removed without leaving a foot-print employing a method for the removal of a selected DNA flanked by two nucleotide sequences in direct repeats.

In addition, methods have been described which allow the design of rare cleaving endonucleases to alter substrate or sequence-specificity of the enzymes, thus allowing to induce a double stranded break at a locus of interest without being dependent on the presence of a recognition site for any of the natural rare-cleaving endonucleases. Briefly, chimeric restriction enzymes can be prepared using hybrids between a zinc-finger domain designed to recognize a specific nucleotide sequence and the non-specific DNA-cleavage domain from a natural restriction enzyme, such as FokI. Such methods have been described e.g. in WO 03/080809, WO94/18313 or WO95/09233 and in Isalan et al., 2001, Nature Biotechnology 19, 656-660; Liu et al. 1997, Proc. Natl. Acad. Sci. USA 94, 5525-5530). Another way of producing custom-made meganucleases, by selection from a library of variants, is described in WO2004/067736. Custom made meganucleases or redesigned meganucleases with altered sequence specificity and DNA-binding affinity may also be obtained through rational design as described in WO2007/047859. Further, WO10/079430, and WO11/072246 describe the design of transcription activator-like effectors (TALEs) proteins with customizable DNA binding specificity and how these can be fused to nuclease domains (e.g. FOKI) to create chimeric restriction enzymes with sequence specificity for basically any DNA sequence, i.e. TALE nucleases (TALENs).

Bedell et al., 2012 (Nature 491:p 114-118) and Chen et al., 2011 (Nature Methods 8:p 753-755) describe oligo-mediated genome editing in mammalian cells using TALENs and ZFNs respectively.

Elliot et al (1998, Mol Cel Biol 18:p 93-101) describes a homology-mediated DSB repair assay wherein the frequency of incorporation of mutations was found to inversely correlate with the distance from the cleavage site.

WO11/154158 and WO11/154159 describe methods and means to modify in a targeted manner the plant genome of transgenic plants comprising chimeric genes wherein the chimeric genes have a DNA element commonly used in plant molecular biology, as well as re-designed meganucleases to cleave such an element commonly used in plant molecular biology.

WO2013026740 describes methods and means are to modify in a targeted manner the genome of a plant in close proximity to an existing elite event using a double stranded DNA break inducing enzyme.

WO2014161821 discloses improved methods and means are provided to modify in a targeted manner the genome of a eukaryotic cell at a predefined site using a double stranded break inducing enzyme such as a TALEN and a donor molecule for repair of the double stranded break.

Recently, a new genome editing method was discovered called Crispr/Cas (Jinek et al., 2012, Science; Gasiunas et al., 2012, PNAS; Cong et al., 2013, Science; Mali et al., 2016, Science; Cho et al., 2013, Nature Biotechnology; Shan et al., 2013, Nature Biotechnology; Nekrasov et al., 2013, Nature Biotechnology; Feng et al., 2013, Cell Res).

WO2014144155 discloses materials and methods for gene targeting using Clustered Regularly Interspersed Short Palindromic Repeats/CRISPR-associated (CRISPR/Cas) systems.

WO2014186686 discloses methods for modifying the genome of plants at a target nucleic acid sequence. Further provided are methods for targeting fusion proteins to target nucleic acid sequences in the genome of plant. Also provided are methods for testing components of the Cas system in plants, modified plants and plant cells, fusion proteins, and nucleic acid molecules encoding such fusion proteins.

WO2014194190 discloses compositions and methods for specific gene targeting and precise editing of DNA sequences in plant genomes using the CRISPR (cluster regularly interspaced short palindromic repeats) associated nuclease. Non-transgenic, genetically modified crops can be produced using these compositions and methods.

WO 2015/026883 discloses compositions and methods for genome modification of a target sequence in the genome of a plant or plant cell, as well as compositions and methods employing a guide polynucleotide/Cas endonuclease system for genome modification of a nucleotide sequence in the genome of a cell or organism, for gene editing, and/or for inserting or deleting a polynucleotide of interest into or from the genome of a cell or organism, breeding methods and methods for selecting plants utilizing a two component RNA guide and Cas endonuclease system and compositions and methods for editing a nucleotide sequence in the genome of a cell.

WO 2015/026885 discloses compositions and methods for genome modification of a target sequence in the genome of a cell, as well as compositions and methods for editing a nucleotide sequence in the genome of a cell and also breeding methods and methods for selecting plants utilizing a two component RNA polynucleotide and Cas endonuclease system.

WO2015/026886 discloses a method for plant genome site-directed modification. Specifically, a method for plant genome site-directed modification introduced by RNA is provided.

WO 2015/048707 discloses materials and methods for conferring geminivirus resistance to plants, and particularly to materials and methods for using CRISPR/Cas systems to confer resistance to geminiviruses to plants.

WO2015117041 discloses methods for generating dominant traits in eukaryotic systems using one or more gene modification-mediated methods are disclosed herein. Aspects of the technology are further directed to methods for silencing SbCSE and/or SbCAD2 gene expression or activity in a sorghum plant.

WO2015131101 discloses novel corn, tomato, and soybean U6, U3, U2, U5, and 7SL snRNA promoters which are useful for CRISPR/Cas-mediated targeted gene modifications in plants. The disclosure also provides methods for use for U6, U3, U2, U5, and 7SL promoters in driving expression of sgRNA polynucleotides which function in a CRISPR/Cas system of targeted gene modification in plants. The disclosure also provides methods of genome modification by insertion of blunt-end DNA fragments at a site of genomic cleavage.

WO2015139008 discloses methods and compositions for making targeted changes to a DNA sequence.

WO2015171894 discloses methods for selecting modified plants with a mutation in a target gene and plants produced by the methods. Specifically, the disclosure provides methods comprising introducing a recombinant expression cassette encoding a genome editing protein into meristematic or germline cells of a parent plant, wherein the genome editing protein specifically recognizes a target gene; crossing or selfing the parent plant, thereby producing a plurality of progeny seeds; and selecting progeny plants grown from the progeny seeds that express a phenotype that can be selected at the intact plant level.

WO2015189693 discloses a viral-mediated genome-editing platform that facilitates multiplexing, obviates stable transformation, and is applicable across plant species. The RNA2 genome of the tobacco rattle virus (TRV) was engineered to carry and systemically deliver a guide RNA molecules into plants overexpressing Cas9 endonuclease.

WO2016007948 discloses compositions and methods for agronomic trait modification of a target sequence in the genome of a plant or plant cell.

WO2016084084 discloses a nucleic acid construct, which comprises a tobacco rattle virus (TRV) sequence and a nucleic acid sequence encoding a single guide RNA (sgRNA) that mediates sequence-specific cleavage in a target sequence of a genome of interest, wherein the TRV sequence is devoid of a functional 2b sequence. Also provided are plant cells comprising the construct and uses of the construct in gene editing.

WO2016106121 discloses methods and compositions for modifying a target site in the genome of a plant cell, whereby such modifications include integration of a transgene and mutations, as well as methods and compositions for identifying and enriching for cells which comprise the modified target site.

WO2016061481 discloses materials and methods to generate numerous small RNAs from one polynucleotide construct (synthetic gene) to facilitate RNA-guided multiplex genome editing, modification, inhibition of expression and other RNA-based technologies.

WO2016116032 discloses a method for conducting site-specific modification in a plant through gene transient expression, comprising the following steps: transiently expressing a sequence-specific nuclease specific to the target fragment in the cell or tissue of the plant of interest, wherein the sequence-specific nuclease is specific to the target site and the target site is cleaved by the nuclease, thereby the site-specific modification of the target site is achieved through DNA repairing of the plant.

Endo et al 2016 (Plant Physiology, February 2016, Vol. 170, pp. 667-677) teaches the sequential delivery with Agrobacterium for first the Cas9 construct optionally with the gRNA followed in a second step by the donor molecule and optionally the gRNA, for enhanced transformation efficiency and to allow sufficient expression of Cas9 at the time when the donor is subsequently introduced.

However, there still remains a need for optimizing genome editing systems in plants, e.g. to increase the recovery of correct editing events. The present invention provides an improved method for making targeted sequence modifications, such as insertions, deletions and replacements, using RGENs and a donor molecule for the introduction of specific modifications, as well as an assay for evaluating genome editing components such as rare-cleaving endonucleases (e.g. RGENs), guide polynucleotides and donor polynucleotides. This will be described hereinafter, in the detailed description, examples and claims.

### SUMMARY

In one aspect, a method is provided for modifying the (nuclear) genome of a plant cell at a preselected site or for producing a plant cell with a modified genome comprising the steps of:
a. introducing into said plant cell an RNA-guided endonuclease (RGEN) and at least one guide polynucleotide, wherein said RGEN and said at least one guide polynucleotide are capable of forming a complex that enables the RGEN to introduce a (double stranded) DNA break or one or more nicks or single stranded breaks, or to induce DNA strand displacement, at or near said preselected site;
b. introducing into said cell at least one donor polynucleotide comprising a polynucleotide of interest;
c. selecting a plant cell wherein said donor polynucleotide has been used as a template for repair of said DNA break, thereby integrating said polynucleotide of interest at said preselected site and resulting in a modification of said genome at said preselected site, wherein said modification is selected from
   i. a replacement of at least one nucleotide;
   ii. a deletion of at least one nucleotide;
   iii. an insertion of at least one nucleotide; or
   iv. any combination of i.-iii.
characterised in that said RGEN, said at least one guide polynucleotide and said at least one donor polynucleotide are introduced into said plant cell by contacting said plant cell with at least one bacterium comprising a chimeric gene encoding said RGEN, at least one chimeric gene encoding said at least one guide polynucleotide and said at least one donor polynucleotide.

The bacterium can be Agrobacterium tumefaciens.

The chimeric gene encoding said endonuclease, said at least one chimeric gene encoding said at least one guide polynucleotide and said at least one donor polynucleotide can be located on one T-DNA vector, such as on one T-DNA molecule (between a single set of T-DNA borders).

In any of the methods described herein, the RGEN can be a nickase or a pair of nickases. The RGEN can be e.g. Cas9, Cpf1, CasX, C2c1, Csm1 (or mutants thereof, such as nicking mutants, or nuclease dead mutants).

The chimeric gene encoding said at least one guide polynucleotide can encode two or more guide polynucleotide sequences.

The coding region of said chimeric gene encoding said endonuclease can be optimized for expression in a plant.

The RGEN can comprise the amino acid sequence of SEQ ID NO 6 from amino acid at position 10 to amino acid at position 1388. The chimeric gene encoding said RGEN can comprise the nucleotide sequence of SEQ ID NO. 5 from nucleotide 28 to nucleotide 4164.

In a further embodiment of any of the methods and compounds/products described herein, the bacterium further comprises a selectable marker gene that is introduced into and expressed in said plant cell. The selectable marker gene can confer upon said plant cell a selectable phenotype. The selectable marker gene can be located on said one T-DNA vector, together with the chimeric gene encoding said RGEN, at least one chimeric gene encoding said at least one guide polynucleotide and said at least one donor polynucleotide. It can be located on the same one T-DNA molecule.

In one embodiment, the modification in the (nuclear) genome resulting from the method confers upon said plant cell a selectable phenotype.

The selectable phenotype, whether conferred by the selectable marker gene or by the modification in the genome at the preselected site, can conveniently be tolerance to one or more herbicides.

In one aspect, the selectable phenotype conferred to said plant cell by said modification can be used for direct selection of a plant cell comprising said modification.

Alternatively, the selectable phenotype conferred to said plant cell by said selectable marker gene can be used to select a plant cell comprising said modification. For this, first one or more plant cells are selected having the selectable phenotype conferred by the selectable marker gene, followed by a selection of a plant cell comprising the intended modification or confirmation of the presence of the intended modification in the selected one or more plant cells.

The plant cell can be comprised within an immature embryo or embryogenic callus.

In any of the methods and compounds/products as described herein, said donor DNA molecule can comprise one or two flanking nucleotide sequences flanking the DNA molecule of interest, said flanking nucleotide sequence or sequences having sufficient homology to the genomic DNA upstream and/or downstream of said preselected site to allow homologous recombination with said upstream and/or downstream DNA region. The polynucleotide of interest may comprise one or more expressible gene(s) of interest, said expressible gene of interest optionally being selected from the group of a herbicide tolerance gene, an insect resistance gene, a disease resistance gene, an abiotic stress resistance gene, an enzyme involved in oil biosynthesis, carbohydrate biosynthesis, an enzyme involved in fiber strength or fiber length, an enzyme involved in biosynthesis of secondary metabolites.

In a further step of the method described herein, the selected plant cell may be grown into a plant comprising said modification. In an even further step, said plant may be crossed with another plant and optionally a progeny plant may be obtained comprising said modification. In an even further step, a progeny plant may be selected that comprises said modification, but does not comprise said chimeric gene encoding said RGEN, said at least one chimeric gene encoding said at least one guide polynucleotide and said selectable marker gene.

The plant cell or plant can be a rice plant cell or plant.

In a further aspect, a plant cell, plant part, seed, plant product or plant comprising a modification at a preselected site in the (nuclear) genome produced according to any of the methods described herein is provided.

Also provided is a bacterium comprising a chimeric gene encoding an RGEN, at least one chimeric gene encoding at least one guide polynucleotide and at least one donor polynucleotide, wherein said bacterium is capable of transferring or introducing said chimeric gene encoding said RGEN, said chimeric gene encoding said guide polynucleotide and said donor polynucleotide into (the nuclear genome of) a plant cell, wherein said RGEN and said guide polynucleotide upon expression in said plant cell are capable of forming a complex that enables the RGEN to introduce a DNA break at a preselected site in the (nuclear) genome of a plant cell and wherein said donor polynucleotide is to be used as a template for repair of said DNA break, according to the methods as described herein. The chimeric gene encoding said RGEN, said chimeric gene encoding said guide polynucleotide and said donor polynucleotide can be located on one vector, such as on one T-DNA molecule (between a pair of T-DNA borders).

Also described is a (T-DNA) vector for use according to the present methods, said vector comprising the chimeric gene encoding an RGEN, the at least one chimeric gene encoding at least one guide polynucleotide and the at least one donor polynucleotide as described herein, e.g. on one T-DNA molecule (between a pair of T-DNA borders).

In another aspect, a method is provided for modifying an endogenous EPSPS gene in a plant cell, or for producing a plant cell having a modified EPSPS gene, or for testing the efficiency of genome editing (components), comprising the steps of:
a. expressing in said cell a site-directed DNA modifying polypeptide recognising a sequence in an endogenous EPSPS gene of said plant and/or introducing into said plant cell a donor polynucleotide that can be used as a template for modifying said endogenous EPSPS gene;
b. evaluating tolerance of said plant cell to one or more EPSPS inhibitors by culturing said plant cell on medium comprising said EPSPS inhibitor(s); and optionally
c. selecting a plant cell having increased tolerance to said EPSPS inhibitor.

An EPSPS inhibitor (e.g. glyphosate) can be used as a direct selective agent.

Further described is a method for modifying the (nuclear) genome of a plant cell at a preselected site comprising the steps of:
a. introducing into said cell a nucleotide-guided DNA modifying polypeptide (NGDMP) and a guide polynucleotide, wherein said NGDMP and guide polynucleotide are capable of forming a complex that enables the NGDMP to modify the genome of a plant cell at a preselected site;
b. selecting a plant cell wherein said genome has been modified at said preselected site characterised in that said NGDMP, said guide polynucleotide are introduced to said plant cell using a particle inflow gun.

Also described is a method for modifying the (nuclear) genome of a plant cell at a preselected site comprising the steps of:
a. introducing into said cell a nucleotide-guided DNA modifying polypeptide (NGDMP) and a guide polynucleotide, wherein said NGDMP and guide polynucleotide are capable of forming a complex that enables the NGDMP to modify the genome of a plant cell at a preselected site;
b. introducing into said cell at least one (plant-expressible) selectable marker gene;
c. selecting one or more plant cells comprising said selectable marker gene;
d. selecting a plant cell wherein said genome has been modified at said preselected site
characterised in that said NGDMP, said at least one guide polynucleotide and said at least one selectable marker gene are introduced into said plant cell by contacting said plant cell with at least one bacterium comprising a chimeric gene encoding said RGEN, at least one chimeric gene encoding said at least one guide polynucleotide and at least one polynucleotide comprising said selectable marker gene.

The RGDMP can be an RGEN, wherein said RGEN and said at least one guide polynucleotide are capable of forming a complex that enables the RGEN to introduce a DNA break at or near said preselected site.

Together with said RGEN and said guide polynucleotide a donor polynucleotide comprising a polynucleotide of interest can be introduced into said plant cell, wherein said donor polynucleotide is used as a template for repair of said DNA break, thereby integrating said polynucleotide of interest at said preselected site and resulting in a modification of said genome at said preselected site.

Also provided is a bacterium comprising a chimeric gene encoding an NGDMP, at least one chimeric gene encoding at least one guide polynucleotide and at least one (plant-expressible) selectable marker gene, wherein said bacterium is capable of transferring or introducing said chimeric gene encoding said NGDMP, said chimeric gene encoding said guide polynucleotide and said selectable marker gene into (the nuclear genome of) a plant cell, wherein said NGDMP and said guide polynucleotide upon expression in said plant cell are capable of forming a complex that enables the NGDMP to modify the (nuclear) genome of a plant cell, according to the herein described methods. The chimeric gene encoding said NGDMP, said chimeric gene encoding said guide polynucleotide and said selectable marker gene can be located on one vector, such as on one T-DNA molecule (between a pair of T-DNA borders).

Further described is a (T-DNA) vector comprising the chimeric gene encoding an NGDMP, the chimeric gene encoding a guide polynucleotide and the selectable marker gene according to the method described herein, such as on one T-DNA molecule (between a pair of T-DNA borders).

### FIGURE LEGENDS

FIG. 1: Schematic overview of the TIPS assay

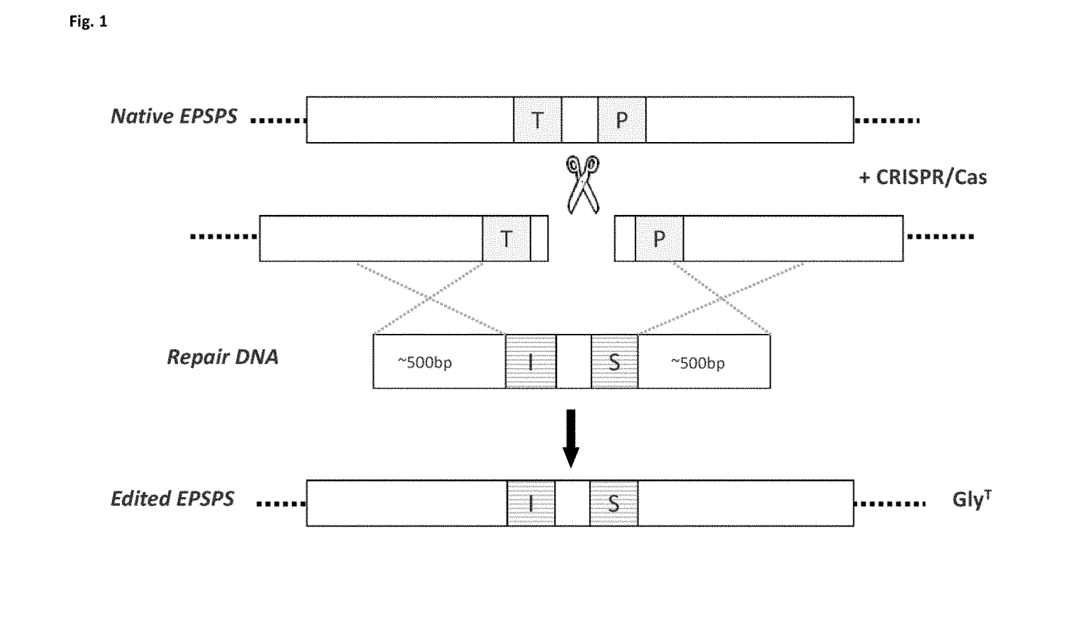

FIG. 2: Alignment of cloned PCR products obtained from GlyT events obtained via bombardment of embryogenic callus

### DETAILED DESCRIPTION

The inventors have found that when transforming plants with a single Agrobacterium strain comprising an RGEN expression cassette (e.g. for Cas9), a chimeric gene encoding a guide polynucleotide and a donor DNA for repair of the induced DNA break, the recovery of precise gene editing events was surprisingly higher than when providing the three components together on separate vectors using direct delivery methods. For this, embryogenic callus or rice immature embryos were transformed with an Agrobacterium strain comprising the three components on one T-DNA vector, more specifically between a single pair of T-DNA borders (i.e. on one T-DNA molecule). When transformed with a donor DNA to introduce a mutation into the rice endogenous EPSPS gene resulting in increased herbicide tolerance (TIPS mutation), this resulted in an increased recovery of glyphosate tolerant events of up to around 10% as compared to direct delivery (about 0.2 to 1.6%). The current Agrobacterium method thus surprisingly resulted in sufficient expression of Cas9 to allow efficient integration of the donor DNA even when introduced simultaneously. The higher frequency of targeted cells by Agrobacterium-mediated DNA delivery compared to particle bombardment is believed to result from DNA introduction into a higher number of cells upon Agrobacterium-mediated DNA delivery compared to DNA delivery by particle bombardment. It was furthermore found that when selecting events based on tolerance provided by a co-delivered selectable marker gene, of the tolerant events almost half (43%) even appeared to contain the desired modification. This clearly enhances the possibility to recover also events of which the intended modification itself is difficult to select for. Agrobacterium mediated transformation (or similar bacterial systems) furthermore has the advantage that it can be used for plant species or varieties that are not amenable to bombardment. Of direct delivery methods, the particle inflow gun bombardment gave the best results. It was furthermore found that by EPSPS targeting, direct glyphosate selection could be used as a readout for successful editing, thus providing a useful assay system for evaluating and comparing genome editing components such as sequence-specific endonucleases (e.g. meganucleases, ZFNs, TALENs, RGENs and the like), guide polynucleotides and donor constructs.

WO2015026883 teaches crossing a plant already containing a Cas9 cassette with plants comprising a gRNA cassette or providing a plant already containing a Cas9 cassette, with a gRNA cassette and optionally a donor construct, thereby pointing towards the importance of already having the RGEN expressed at the time of introducing the guide and donor polynucleotide. WO2015026883 furthermore teaches EPSPS editing in maize by direct delivery (particle gun bombardment), and using bialaphos selection for the enrichment of editing events resulting from a co-transformed moPAT selectable marker gene (indirect selection). After a long selection and culturing period, starting from 3282 embryos, 390 TO plants were produced, of which 72% contained mutagenized EPSPS. Most modified EPSPS events however resulted from the NHEJ while only a small fraction of the modified events contained the intended TIPS mutation from the recombination template (donor nucleic acid).

WO2015/026886 describes maize EPSPS editing wherein the recombination template was co-delivered with the sgRNA expression cassette and a Cas9 expression vector using particle bombardment together with the moPAT selectable marker gene and initial selection was done on bialaphos.

WO2015131101 described codelivery of the three components using PEG transformation and bombardment. WO2016007948 discloses co-delivery of a gRNA construct, the polynucleotide modification template, a Cas9 cassette by particle bombardment.

Endo et al 2016 (Plant Physiology, February 2016, Vol. 170, pp. 667-677) teaches the sequential delivery with Agrobacterium for first the Cas9 construct optionally with the gRNA followed in a second step by the donor molecule and optionally the gRNA, for enhanced transformation efficiency and to allow sufficient expression of Cas9 at the time when the donor is subsequently introduced.

Accordingly, the prior art discloses simultaneous delivery of the gRNA construct, RGEN construct and donor polynucleotide by direct delivery methods, or the prior delivery of at least the RGEN construct only later followed by the donor polynucleotide.

Thus, in a first aspect, the invention relates to a method for modifying the (nuclear) genome of a plant cell at a preselected site or for producing a plant cell comprising a modification at a preselected site in its (nuclear) genome (i.e. a targeted modification), comprising the steps of:
a. introducing into said plant cell an RNA-guided endonuclease (RGEN) and at least one guide polynucleotide, wherein said RGEN and said at least one guide polynucleotide are capable of forming a complex that enables the RGEN to introduce a DNA break (a double stranded DNA break, or one or more nicks or single stranded breaks, or to induce strand displacement (e.g by a catalytically inactive nuclease) at or near said preselected site;
b. introducing into said cell at least one donor polynucleotide comprising a polynucleotide of interest;
c. selecting a plant cell wherein said donor polynucleotide has been used as a template for repair of said DNA break, thereby integrating said polynucleotide of interest at said preselected site and resulting in a modification of said genome at said preselected site, wherein said modification is selected from
   i. a replacement of at least one nucleotide i.e. one or more nucleotides;
   ii. a deletion of at least one nucleotide i.e. one or more nucleotides;
   iii. an insertion of at least one nucleotide i.e. one or more nucleotides; or
   iv. any combination of i.-iii.
      characterised in that said RGEN, said at least one guide polynucleotide and said at least one donor polynucleotide are introduced into said plant cell by contacting said cell with at least one bacterium, said at least one bacterium comprising a chimeric gene encoding said RGEN, at least one chimeric gene encoding said at least one guide polynucleotide and said at least one donor polynucleotide.

An RNA-guided nuclease or endonuclease, as used herein, is an RNA-guided DNA modifying polypeptide having (endo)nuclease activity.

RGENs are typically derived from CRISPR systems, which are a widespread class of bacterial systems for defense against foreign nucleic acid. CRISPR systems are found in a wide range of eubacterial and archaeal organisms. CRISPR systems include type I, II, III and V sub-types (see e.g. 2007025097; WO2013098244; WO2014022702; WO2014093479; WO2015155686; EP3009511; US2016208243). Wild-type type II CRISPR/Cas systems utilize an RNA-guided nuclease, e.g. Cas9, in complex with guide and activating RNA to recognize and cleave foreign nucleic acid (Jinek et al., 2012, supra).

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Further Cas9 proteins, homologs and variants thereof and methods for use in genome editing or are described in, e.g., Chylinksi, et al., RNA Biol. 2013 May 1; 10(5): 726-737; Nat. Rev. Microbiol. 2011 June; 9(6): 467-477; Hou, et al., Proc Natl Acad Sci USA. 2013 Sep 24; 110(39):15644-9; Sampson et al., Nature. 2013 May 9; 497(7448):254-7; and Jinek, et al., Science. 2012 Aug. 17; 337(6096):816-21; WO2013142578; WO2013176772; WO2014065596; WO2014089290; WO2014093709; WO2014093622; WO2014093655; WO2014093701; WO2014093712; WO2014093635; WO2014093595; WO2014093694; WO2014093661; WO2014093718; WO2014093709; WO2014099750; WO2014113493; WO2014190181; WO2015006294; WO2015071474; WO2015077318; WO2015089406; WO2015103153; WO201621973; WO201633298; WO201649258, all incorporated herein by reference).

Further RNA-guided nucleases include e.g. Cpf1 (also known as Cas12a) and homologues and variants thereof (as e.g. described in Zetsche et al., Cell, Volume 163, Issue 3, p 759-771, 22 Oct. 2015; EP3009511; US2016208243; Kleinstiver et al., Nat Biotechnol. 2016 August; 34(8):869-74; Gao et al., Cell Res. 2016 August; 26(8):901-13; Hur et al., 2016 Nat Biotech, Kim et al., 2016 Nat Biotech; Yamano et al., Cell. Apr. 21, 2016; WO2016166340; WO2016205711, further Broad, WO2017064546, WO2017141173, WO201795111, all incorporated herein by reference).

Even further RNA-guided nucleases include e.g. C2c1 and C2c3 (also known as Cas12b and Cas12c respectively; Shmakov et al., Mol Cell. 2015 Nov. 5; 60(3):385-97; EP3009511; WO2016205749), Csm1 (WO2017141173), CasX and CasY (Burnstein et al., Nature vol 542, 2017), and further nucleases from additional class 2 crispr systems (Schmakov et al., Nature Reviews Microbiology 15, 169-182, 2017) (all incorporated herein by reference).

Further RNA-guided nucleases can include Argonaut-like proteins, as eg described in WO2015157534.

Further RNA-guided nucleases and other RNA-guided polypeptides are described in WO2013088446.

In one embodiment, the RGEN can also be an RNA-guided nicking enzyme (nickase), or a pair of RNA-guided nicking enzymes, that each introduce a break in only one strand of the double stranded DNA at or near the preselected site. Of a pair of nickases, the one enzyme introduces a break in one strand of the DNA at or near the preselected site, while the other enzyme introduces a break in the other strand of the DNA at or near the preselected site. The two single-stranded breaks can be introduced at the same nucleotide position on both strands, resulting in a blunt ended double stranded DNA break, but the two single stranded breaks can also be introduced at different nucleotide positions in each strand, resulting in a 5′ or 3′ overhang at the break site (“sticky ends” or “staggered cut”). In one embodiment, the two guide polynucleotides directing the nickases are chosen in such as way as to create a break with a 3′ overhang, as e.g. described in WO201628682. Nicking mutants and uses thereof are e.g. described in the above documents and specifically in WO2014191518, WO2014204725, WO201628682. Also a single nicking mutant, which introduced a break in only one of the two strands of the DNA (i.e. a single-stranded DNA break), can enhance homology directed repair (HDR) with a donor polynucleotide (Richardson et al. 2016, Nature Biotechnology 34, 339-344; U.S. 62/262,189).

As an alternative to a nuclease or nickase, also nuclease deficient (also referred to as “dead” or catalytically inactive) variants of the above described nucleases, such as dCas9, can be used to increase targeted insertion of a donor polynucleotide, as e.g. described in Richardson et al. 2016, Nature Biotechnology 34, 339-344; U.S. 62/262,189). Such variants lack the ability to cleave or nick DNA but are capable of being targeted to and bind DNA (see e.g. WO2013176772, EP3009511). These “dead” nucleases are believed to induce strand displacement by binding to one of the two strands (“DNA melting”), thereby enhancing recombination with the donor polynucleotide by allowing the donor polynucleotide to anneal with the other “free” DNA strand.

Nicking mutants have been described of various RGENs and involve one or more mutations in a catalytic domain, such as the HNH and RuvC domains (e.g. Cas9) of the RuvC-like domain (e.g. Cpf1). For example, SpCas9 can be converted into a nickase by mutating D10A in the RuvC and 863A in the HNH nuclease domain converts SpCas9 into a DNA nickase, while inactivation of both nuclease domain results in a catalytically inactive protein (Jinek et al., 2012, supra, Gasiunas et al., 2012, Proc. Natl. Acad. Sci. USA 109, E2579-E2586). In Cpf1, it was found that the D917A as well as the E1006A mutation completely inactivated the DNA cleavage activity of FnCpf1, and while D1255A significantly reduced nucleolytic activity (Zetsche et al., 2015, supra). Corresponding residues other RGEN (e.g. Cas9 or Cpf1) variants can be determined by optimal alignment.

A chimeric gene encoding an RGEN, as used herein, typically comprises the following operably linked-components: a DNA region coding for the RGEN (RGEN coding region), a (plant-expressible) promoter and optionally a polyadenylation and transcription terminator (3′ end region) functional in plants. Such a promoter can be a constitutive promoter, but depending on when RGEN expression is desired also other promoters can be used such as inducible promoters (e.g. stress-inducible promoters, drought-inducible promoters, hormone-inducible promoters, chemical-inducible promoters, etc.), tissue-specific promoters, developmentally regulated promoters and the like.

A plant-expressible constitutive promoter, is a promoter capable of directing high levels of expression in most cell types (in a spatio-temporal independent manner). Examples of plant expressible constitutive promoters include promoters of bacterial origin, such as the octopine synthase (OCS) and nopaline synthase (NOS) promoters from Agrobacterium, but also promoters of viral origin, such as that of the cauliflower mosaic virus (CaMV) 35S transcript (Hapster et al., 1988, Mol. Gen. Genet. 212: 182-190) or 19S RNAs genes (Odell et al., 1985, Nature. 6; 313(6005):810-2; U.S. Pat. No. 5,352,605; WO 84/02913; Benfey et al., 1989, EMBO J. 8:2195-2202), the enhanced 2×35S promoter (Kay at al., 1987, Science 236:1299-1302; Datla et al. (1993), Plant Sci 94:139-149) promoters of the cassava vein mosaic virus (CsVMV; WO 97/48819, U.S. Pat. No. 7,053,205), 2×CsVMV (WO2004/053135) the circovirus (AU 689 311) promoter, the sugarcane bacilliform badnavirus (ScBV) promoter (Samac et al., 2004, Transgenic Res. 13(4):349-61), the figwort mosaic virus (FMV) promoter (Sanger et al., 1990, Plant Mol Biol. 14(3):433-43), the subterranean clover virus promoter No 4 or No 7 (WO 96/06932) and the enhanced 35S promoter as described in U.S. Pat. Nos. 5,164,316, 5,196,525, 5,322,938, 5,359,142 and 5,424,200. Among the promoters of plant origin, mention will be made of the promoters of the plant ribulose-biscarboxylase/oxygenase (Rubisco) small subunit promoter (U.S. Pat. No. 4,962,028; WO99/25842) from zea mays and sunflower, the promoter of the Arabidopsis thaliana histone H4 gene (Chabouté et al., 1987), ubiquitin promoters (Holtorf et al., 1995, Plant Mol. Biol. 29:637-649, U.S. Pat. No. 5,510,474) of Maize, Rice and sugarcane, the Rice actin 1 promoter (Act-1, U.S. Pat. No. 5,641,876), the histone promoters as described in EP 0 507 698 A1, the Maize alcohol dehydrogenase 1 promoter (Adh-1) (from http://www.patentlens.net/daisy/promoters/242.html)). Also the small subunit promoter from Chrysanthemum may be used if that use is combined with the use of the respective terminator (Outchkourov et al., Planta, 216: 1003-1012, 2003).

Further plant expressible-promoters can be plant gene promoters that regulate gene expression in response to environmental, hormonal, chemical, developmental signals, and in a tissue- or cell- or germline- or developmental stage-specific manner. Choice of a promoter is based largely on the phenotype of interest and is determined by such factors as tissue (e.g., seed, fruit, root, pollen, vascular tissue, flower, carpel, etc.), inducibility (e.g., in response to wounding, heat, cold, drought, light, pathogens, etc.), timing, developmental stage, and the like.

Additional promoters that can be used to practice this invention are those that elicit expression in response to stresses, such as the RD29 promoters that are activated in response to drought, low temperature, salt stress, or exposure to ABA (Yamaguchi-Shinozaki et al., 2004, Plant Cell, Vol. 6, 251-264; WO12/101118), but also promoters that are induced in response to heat (e.g., see Ainley et al. (1993) Plant Mol. Biol. 22: 13-23), light (e.g., the pea rbcS-3A promoter, Kuhlemeier et al. (1989) Plant Cell 1: 471-478, and the maize rbcS promoter, Schaffher and Sheen (1991) Plant Cell 3: 997-1012); wounding (e.g., wunl, Siebertz et al. (1989) Plant Cell 1: 961-968); pathogens (such as the PR-I promoter described in Buchel et al. (1999) Plant Mol. Biol. 40: 387-396, and the PDF 1.2 promoter described in Manners et al. (1998) Plant Mol. Biol. 38: 1071-1080), and chemicals such as methyl jasmonate or salicylic acid (e.g., see Gatz (1997) Annu. Rev. Plant Physiol. Plant Mol. Biol. 48: 89-108). In addition, the timing of the expression can be controlled by using promoters such as those acting at senescence (e.g., see Gan and Amasino (1995) Science 270: 1986-1988); or late seed development (e.g., see Odell et al. (1994) Plant Physiol. 106: 447-458).

Use may also be made of salt-inducible promoters such as the salt-inducible NHX1 promoter of rice landrace Pokkali (PKN) (Jahan et al., 6th International Rice Genetics symposium, 2009, poster abstract P4-37), the salt inducible promoter of the vacuolar H+-pyrophosphatase from Thellungiella halophila (TsVP1) (Sun et al., BMC Plant Biology 2010, 10:90), the salt-inducible promoter of the Citrus sinensis gene encoding phospholipid hydroperoxide isoform gpx1 (Avsian-Kretchmer et al., Plant Physiology July 2004 vol. 135, p 1685-1696).

Tissue-specific and/or developmental stage-specific promoters are used, e.g., promoter that can promote transcription only within a certain time frame of developmental stage within that tissue. See, e.g., Blazquez (1998) Plant Cell 10:791-800, characterizing the Arabidopsis LEAFY gene promoter. See also Cardon (1997) Plant J 12:367-77, describing the transcription factor SPL3, which recognizes a conserved sequence motif in the promoter region of the A. thaliana floral meristem identity gene API; and Mandel (1995) Plant Molecular Biology, Vol. 29, pp 995-1004, describing the meristem promoter eIF4. Tissue specific promoters which are active throughout the life cycle of a particular tissue can be used. In one aspect, the nucleic acids of the invention are operably linked to a promoter active primarily only in cotton fiber cells, in one aspect, the nucleic acids of the invention are operably linked to a promoter active primarily during the stages of cotton fiber cell elongation, e.g., as described by Rinehart (1996) supra. The nucleic acids can be operably linked to the Fbl2A gene promoter to be preferentially expressed in cotton fiber cells (Ibid). See also, John (1997) Proc. Natl. Acad. Sci. USA 89:5769-5773; John, et al., U.S. Pat. Nos. 5,608,148 and 5,602,321, describing cotton fiber-specific promoters and methods for the construction of transgenic cotton plants. Root-specific promoters may also be used to express the nucleic acids of the invention. Examples of root-specific promoters include the promoter from the alcohol dehydrogenase gene (DeLisle (1990) Int. Rev. Cytol. 123:39-60) and promoters such as those disclosed in U.S. Pat. Nos. 5,618,988, 5,837,848 and 5,905,186. Other promoters that can be used to express the nucleic acids of the invention include, e.g., ovule-specific, embryo-specific, endosperm-specific, integument-specific, seed coat-specific promoters, or some combination thereof; a leaf-specific promoter (see, e.g., Busk (1997) Plant J. 11:1285 1295, describing a leaf-specific promoter in maize); the ORF 13 promoter from Agrobacterium rhizogenes (which exhibits high activity in roots, see, e.g., Hansen (1997) supra); a maize pollen specific promoter (see, e.g., Guerrero (1990) Mol. Gen. Genet. 224:161 168); a tomato promoter active during fruit ripening, senescence and abscission of leaves, a guard-cell preferential promoter e.g. as described in PCT/EP12/065608, and, to a lesser extent, of flowers can be used (see, e.g., Blume (1997) Plant J. 12:731 746); a pistil-specific promoter from the potato SK2 gene (see, e.g., Ficker (1997) Plant Mol. Biol. 35:425 431); the Blec4 gene from pea, which is active in epidermal tissue of vegetative and floral shoot apices of transgenic alfalfa making it a useful tool to target the expression of foreign genes to the epidermal layer of actively growing shoots or fibers; the ovule-specific BELI gene (see, e.g., Reiser (1995) Cell 83:735-742, GenBank No. U39944); and/or, the promoter in Klee, U.S. Pat. No. 5,589,583, describing a plant promoter region is capable of conferring high levels of transcription in meristematic tissue and/or rapidly dividing cells. Further tissue specific promoters that may be used according to the invention include: seed-specific promoters (such as the napin, phaseolin or DC3 promoter described in U.S. Pat. No. 5,773,697), fruit-specific promoters that are active during fruit ripening (such as the dru 1 promoter (U.S. Pat. No. 5,783,393), or the 2AI 1 promoter (e.g., see U.S. Pat. No. 4,943,674) and the tomato polygalacturonase promoter (e.g., see Bird et al. (1988) Plant Mol. Biol. 11: 651-662), flower-specific promoters (e.g., see Kaiser et al. (1995) Plant Mol. Biol. 28: 231-243), pollen-active promoters such as PTA29, PTA26 and PTAI 3 (e.g., see U.S. Pat. No. 5,792,929) and as described in e.g. Baerson et al. (1994 Plant Mol. Biol. 26: 1947-1959), promoters active in vascular tissue (e.g., see Ringli and Keller (1998) Plant Mol. Biol. 37: 977-988), carpels (e.g., see Ohl et al. (1990) Plant Cell 2), pollen and ovules (e.g., see Baerson et al. (1993) Plant Mol. Biol. 22: 255-267). In alternative embodiments, plant promoters which are inducible upon exposure to plant hormones, such as auxins, are used to express the nucleic acids used to practice the invention. For example, the invention can use the auxin-response elements El promoter fragment (AuxREs) in the soybean {Glycine max L.) (Liu (1997) Plant Physiol. 115:397-407); the auxin-responsive Arabidopsis GST6 promoter (also responsive to salicylic acid and hydrogen peroxide) (Chen (1996) Plant J. 10: 955-966); the auxin-inducible parC promoter from tobacco (Sakai (1996) 37:906-913); a plant biotin response element (Streit (1997) Mol. Plant Microbe Interact. 10:933-937); and, the promoter responsive to the stress hormone abscisic acid (ABA) (Sheen (1996) Science 274:1900-1902). Further hormone inducible promoters that may be used include auxin-inducible promoters (such as that described in van der Kop et al. (1999) Plant Mol. Biol. 39: 979-990 or Baumann et al., (1999) Plant Cell 11: 323-334), cytokinin-inducible promoter (e.g., see Guevara-Garcia (1998) Plant Mol. Biol. 38: 743-753), promoters responsive to gibberellin (e.g., see Shi et al. (1998) Plant Mol. Biol. 38: 1053-1060, Willmott et al. (1998) Plant Molec. Biol. 38: 817-825) and the like.

Other promoters may be plant promoters which are inducible upon exposure to chemicals reagents which can be applied to the plant, such as herbicides or antibiotics. For example, the maize In2-2 promoter, activated by benzenesulfonamide herbicide safeners, can be used (De Veylder (1997) Plant Cell Physiol. 38:568-577); application of different herbicide safeners induces distinct gene expression patterns, including expression in the root, hydathodes, and the shoot apical meristem. Coding sequence can be under the control of, e.g., a tetracycline-inducible promoter, e.g., as described with transgenic tobacco plants containing the Avena sativa L. (oat) arginine decarboxylase gene (Masgrau (1997) Plant J. 11:465-473); or, a salicylic acid-responsive element (Stange (1997) Plant J. 11:1315-1324). Using chemically-{e.g., hormone- or pesticide-) induced promoters, i.e., promoter responsive to a chemical which can be applied to the transgenic plant in the field, expression of a polypeptide of the invention can be induced at a particular stage of development of the plant. Use may also be made of the estrogen-inducible expression system as described in U.S. Pat. No. 6,784,340 and Zuo et al. (2000, Plant J. 24: 265-273) to drive the expression of the nucleic acids used to practice the invention.

In alternative embodiments, a promoter may be used whose host range is limited to target plant species, such as corn, rice, barley, wheat, potato or other crops, inducible at any stage of development of the crop.

In alternative embodiments, a tissue-specific plant promoter may drive expression of operably linked sequences in specific target tissues. In alternative embodiments, a tissue-specific promoter that drives expression preferentially in the target tissue or cell type, but may also lead to some expression in other tissues as well, is used.

In alternative embodiments, use may be made of promoter elements as e.g. described on http://arabidopsis.med.ohio-state.edu/AtcisDB/bindingsites.html., which in combination should result in a functional promoter.

RNA-guided proteins, such as RGENs, are targeted to a specific target nucleic acid, e.g. a DNA, by means of a guide polynucleotide, such as a guide RNA. A guide polynucleotide, as used herein, is a polynucleotide that can direct an RNA guided protein such as an RGEN, to a specific target sequence. A preferred guide polynucleotide is a guide RNA or gRNA. A “target sequence” refers to a sequence to which a guide sequence is designed to target, e.g. have complementarity. The skilled person will be well aware of the requirements of guide polynucleotides to be used in conjunction with certain RGEN, as well as certain requirements for the target sequence when using certain RGENs (e.g. the specific protospacer adjacent motive “PAM”), as also describe in the above cited documents. Likewise, the skilled person would be well aware of the location of the cleavage site of the respective RGEN within the target sequence.

At least one guide polynucleotide, as used herein, refers to one or more guide polynucleotides. Indeed, constructs can be provided to the plant cell for expression of more than one guide polynucleotide, so as to allow multiplexing, i.e. targeting multiple sites simultaneously. Such methods are e.g. described in Xie et al. (PNAS, Mar. 17, 2015, vol. 112, no. 11, p 3570-3575), WO2016061481, WO2015099850, Char et al., (Plant Biotechnology Journal, 5 Sep. 2016) (incorporated herein by reference). The composition and structure of guide RNAs (including potentially tracr and PAM regions) has been well described in the art and guide polynucleotides described herein correspond to those described in the art.

A chimeric gene encoding said at least one guide polynucleotide (guide RNA) comprises the following operably linked elements, a promoter suitable for the expression of an RNA, a DNA region encoding the gRNA and optionally a termination region (′3 end region or terminator) suitable for the expression of an RNA. Such promoters are typically DNA polymerase III (pol III) promoters, but also pol II promoters can be used (WO2015099850). Plant-expressible pol III promoters are particularly suitable for expression of the guide RNA according to the present invention, as are plant-functional pol III terminators as e.g. described in Jiang W. et al., 2013; WO2014186686; WO2014194190; WO2015026883; WO2015026885; WO2015026886; WO2015131101; WO2015171894; WO2016007948.

A donor polynucleotide, as used herein, refers to a polynucleotide (e.g. a single-stranded or double-stranded DNA molecule or RNA molecule) that is used as a template for modification of the genomic DNA at the preselected site in the vicinity of or at the cleavage site, i.e. the site of the DNA break, and is hence also referred to as recombination template. As used herein, “use as a template for modification of the genomic DNA”, means that (part of the) the donor polynucleotide is copied or integrated at the preselected site. This can be by homologous recombination between homologous sequences in the donor polynucleotide and sequences in the vicinity of the preselected site, or optionally in combination with non-homologous end-joining (NHEJ) at one of the two ends of the donor polynucleotides, thereby resulting in the incorporation of the polynucleotide of interest at the preselected site. Integration by homologous recombination will allow precise joining of the donor polynucleotide with the target genome up to the nucleotide level, while NHEJ may result in small insertions/deletions at the junction between the donor polynucleotide and genomic DNA.

A polynucleotide of interest, as used herein, refers to a sequence in the donor polynucleotide that upon copying or integration into the target genome results in the intended, targeted modification, which is also referred to as a precise or exact editing event or targeted insertion event. The modification can be a replacement of at least one nucleotide, a deletion of at least one nucleotide, an insertion of at least one nucleotide, or any combination thereof, as long as the resulting sequence differs in at least one nucleotide from the original genomic sequence. Accordingly, the modification can be at least one nucleotide change but also multiple nucleotide changes, such as replacements, insertions or deletions or combinations thereof, thereby allowing the identification of the modification by techniques well known in the art, such as sequencing, PCR analysis, restriction analysis and the like.

As used herein “a preselected site” or “predefined site” indicates a particular nucleotide sequence in the genome (e.g. the nuclear genome) at which location it is desired to insert, replace and/or delete one or more nucleotides. This can e.g. be an endogenous locus or a particular nucleotide sequence in or linked to a previously introduced foreign DNA or transgene. The preselected site can be a particular nucleotide position at (after) which it is intended to make an insertion of one or more nucleotides. The preselected site can also comprise a sequence of one or more nucleotides which are to be exchanged (replaced) or deleted.

As used herein “at or near said preselected site”, with respect to the location of site of the DNA break induction, refers to the break site (cleavage site) overlapping with the preselected site (at) or being located further away from (near or in the vicinity the preselected site, i.e. the site at which the targeted modification takes place. This can be e.g. 1 bp, 2 bp, 3 bp, 4 bp, 5 bp, 6 bp, 7 bp, 8 bp, 9 bp, 10 bp, 15, bp, 20 bp, 25 bp. 30 bp, 40 bp, 50 bp from the preselected site, but also e.g. 100 bp, 200 bp, 300 bp, 400 bp, 500 bp, 1 kb, 2 kb or 5 kb, as e.g. described in WO2014161821.

A bacterium according to the present invention can be any bacterium, preferably non-pathogenic or disarmed (not containing oncogenes), that is capable of directing the transfer of DNA contained within the bacterium stably into the genome of a plant cell. Such bacteria harbor one or more plasmids, e.g. a tumor-inducing plasmis (Ti plasmid) or a root-inducing plasmid (Ri plasmid), of which the so-called transfer DNA (T-DNA) is transferred into the plant cell and incorporated into the plant genome following transformation. Certain soil bacteria of the order of the Rhizobiales have this capacity, such as Rhizobiaceae (e.g. Rhizobium spp., Sinorhizobium spp., Agrobacterium spp); Phyllobacteriaceae (e.g. Mesorhizobium spp., Phyllobacterium spp.); Brucellaceae (e.g. Ochrobactrum spp.); Bradyrhizobiaceae (e.g. Bradyrhizobium spp.), and Xanthobacteraceae (e.g. Azorhizobium spp.), Agrobacterium spp., Rhizobium spp., Sinorhizobium spp., Mesorhizobium spp., Phyllobacterium spp. Ochrobactrum spp. and Bradyrhizobium spp., examples of which include Ochrobactrum sp., Rhizobium sp., Mesorhizobium loti, Sinorhizobium meliloti. Examples of Rhizobia include R. leguminosarum by, trifolii, R. leguminosarum bv,phaseoli and Rhizobium leguminosarum, by, viciae (U.S. Pat. No. 7,888,552).

Other bacteria that can be employed to carry out the invention which are capable of transforming plants cells and induce the incorporation of DNA into the plant genome are bacteria of the genera Azobacter (aerobic), Closterium (strictly anaerobic), Klebsiella (optionally aerobic), and Rhodospirillum (anaerobic, photosynthetically active). Transfer of a Ti plasmid was also found to confer tumor inducing ability on several Rhizobiaceae members such as Rhizobium trifolii, Rhizobium leguminosarum and Phyllobacterium myrsinacearum, while Rhizobium sp. NGR234, Sinorhizobium meliloti and Mesorhizobium loti could indeed be modified to mediate gene transfer to a number of diverse plants (Broothaerts et al., 2005, Nature, 433:629-633).

The mechanism of T-DNA transfer to plant cells by Agrobacterium and the like has been well documented (see e.g. Tzfira and Citovsky (2006) Curr. Opin. Biotechnol. 17: 147-154; Gelvin (2003) Microbiol. Molec. Biol. Rev. 67: 16-37; Gelvin (2009) Plant Physiol. 150: 1665-1676). Briefly, a T-DNA is typically delimited by two border regions, referred to as right border (RB) and left border (LB). The borders are nicked by virulence protein VirD2 which produces single stranded transferred DNA (the “T-strand”) with covalent attachment of the 40 VirD2 on its 5′ end. The protein-DNA complex, also including Agrobacterium VirE2 protein, exits Agrobacterium cells through the so-called Type 4 secretion system (T4SS, both virulence protein and ssDNA transporter), and is transferred into plant cells and integrated in the plant genome with the help of both Agrobacterium virulence proteins and plant factors. The vir genes are normally found as a series of operons on the Ti or Ri plasmids. Various Ti and Ri plasmids differ somewhat in the complement of vir genes, with, for example, virF not always being present. The use of Agrobacterium-mediated vectors to introduce DNA into plant cells is well known in the art. See, for example, Fraley et al., (1985; Biotechnology 3: 629-635), Rogers et al., (1987; Methods Enzymol 153: 253-277) and U.S. Pat. No. 5,563,055.

The Left Border (LB) is not strictly required for T-DNA transfer, as oncogene containing T-DNAs lacking the LB but containing the RB were highly virulent whereas such T-DNAs containing the LB but not the RB were completely avirulent (Jen et al., 1986, J Bacteriol 166:491-499). Thus, a T-DNA, as used herein, refers to a DNA molecule that is transferable to a plant cell by a bacterium, which comprises in addition to the DNA to be used for repair of the DNA break (the repair DNA) at least one T-DNA border, preferably at least the right T-DNA border. However, to prevent incorporation of undesired vector elements, the left and the right border should both be included, i.e flanking the DNA of interest, as these define the ends of the T-DNA molecules.

It has been described that the left border is more prone to “read through” than the right border (ref). Thus, in order to reduce the chance of two DNAs in one vector being processed as a single T-DNA molecule, the two T-DNAs can be oriented such that at the point on the vector where the two T-DNAs are located closest to each other, there are no two left borders facing each other (head to head; RB-LB; LB-RB). Thus, in one embodiment, the orientation of the two T-DNAs on the vector is such that at the point on the vector where the two T-DNAs are located closest to each other, there are two right borders facing each other (the T-DNAs are in a tail to tail orientation: LB-RB; RB-LB). In a more preferred embodiment, the orientation of the two T-DNAs on the vector is in the same direction, such that the left border of the one T-DNA faces the right border of the other T-DNA, i.e the two T-DNAs are in a head to tail orientation (LB-LB; RB-LB).

Examples of the bacterium belonging to the genus Agrobacterium which may be employed for the invention include but is not limited to Agrobacterium tumefaciens, Agrobacterium rhizogenes, Agrobacterium radiobacter, Agrobacterium rubi, Argobacterium vitis. The Agrobacterium species used can be a wild type (e.g., virulent) or a disarmed strain. Suitable strains of Agrobacterium include wild type strains (e.g., such as Agrobacterium tumefaciens) or strains in which one or more genes is mutated to increase transformation efficiency, e.g., such as Agrobacterium strains wherein the vir gene expression and/or induction thereof is altered due to the presence of mutant or chimeric virA or virG genes (e.g. Chen and Winans, 1991, J. Bacteriol. 173: 1139-1144; and Scheeren-Groot et al., 1994, J. Bacteriol. 176:6418-6246), Agrobacterium strains comprising an extra virG gene copy, such as the super virG gene derived from pTiBo542, preferably linked to a multiple-copy plasmid, as described in U.S. Pat. No. 6,483,013, for example. Other suitable strains include, but are not limited to: A. tumefaciens GV3101 (pMP90)) (Konc and Schell, 1986, Mol Gen Genet. 204:383-396), LBA4404 (Hoekema et al., Nature 303: 179-180 (1983)); EHA101 (Hood et al., J. Bac. 168: 1291-1301 (1986)); EHA105 (Hood et al., Trans Res. 2: 208-218 (1993)); AGL1 (Lazo et al., Bio Technology 2: 963-967 (1991)).

For Agrobacterium-mediated plant transformation, the DNA to be inserted into the plant cell can be cloned into special plasmids, for example, either into an intermediate (shuttle) vector or into a binary vector. Intermediate vectors are not capable of independent replication in Agrobacterium cells, but can be manipulated and replicated in common Escherichia coli molecular cloning strains. Such intermediate vectors comprise sequences are commonly framed by the right and left T-DNA border repeat regions, that may include a selectable marker gene functional for the selection of transformed plant cells, a cloning linker, a cloning polylinker, or other sequence which can function as an introduction site for genes destined for plant cell transformation. Cloning and manipulation of genes desired to be transferred to plants can thus be easily performed by standard methodologies in E. coli, using the shuttle vector as a cloning vector. The finally manipulated shuttle vector can subsequently be introduced into Agrobacterium plant transformation strains for further work. The intermediate shuttle vector can be transferred into Agrobacterium by means of a helper plasmid (via bacterial conjugation), by electroporation, by chemically mediated direct DNA transformation, or by other known methodologies. Shuttle vectors can be integrated into the Ti or Ri plasmid or derivatives thereof by homologous recombination owing to sequences that are homologous between the Ti or Ri plasmid, or derivatives thereof, and the intermediate plasmid. This homologous recombination (i.e. plasmid integration) event thereby provides a means of stably maintaining the altered shuttle vector in Agrobacterium, with an origin of replication and other plasmid maintenance functions provided by the Ti or Ri plasmid portion of the co-integrant plasmid. The Ti or Ri plasmid also comprises the vir regions comprising vir genes necessary for the transfer of the T-DNA. The plasmid carrying the vir region is commonly a mutated Ti or Ri plasmid (helper plasmid) from which the T-DNA region, including the right and left T-DNA border repeats, have been deleted. Such pTi-derived plasmids, having functional vir genes and lacking all or substantially all of the T-region and associated elements are descriptively referred to herein as helper plasmids.

T-DNA vectors for plant transformation can also be prepared using the so-called superbinary system. This is a specialized example of the shuttle vector/homologous recombination system (reviewed by Komari et al, (2006) In: Methods in Molecular Biology (K. Wang, ed.) No. 343: Agrobacterium Protocols (2nd Edition, Vol. 1) HUMANA PRESS Inc., Totowa, N.J., pp. 15-41; and Komori et al, (2007) Plant Physiol. 145: 1155-1160). The Agrobacterium tumefaciens host strain employed with the superbinary system is LBA4404(pSBI). Strain LBA4404(pSBI) harbors two independently-replicating plasmids, pAL4404 and pSBI. pAL4404 is a Ti-plasmid-derived helper plasmid which contains an intact set of vir genes (from Ti plasmid pTiACH5), but which has no T-DNA region (and thus no T-DNA left and right border repeat sequences). Plasmid pSBI supplies an additional partial set of vir genes derived from pTiBo542; this partial vir gene set includes the virB operon and the virC operon, as well as genes virG and virDI. One example of a shuttle vector used in the superbinary system is pSBI I, which contains a cloning polylinker that serves as an introduction site for genes destined for plant cell transformation, flanked by right and left T-DNA border repeat regions. Shuttle vector pSBI 1 is not capable of independent replication in Agrobacterium, but is stably maintained as a co-integrant plasmid when integrated into pSBI by means of homologous recombination between common sequences present on pSBI and pSBI I. Thus, the fully modified T-DNA region introduced into LBA4404(pSBI) on a modified pSBI I vector is productively acted upon and transferred into plant cells by Vir proteins derived from two different Agrobacterium Ti plasmid sources (pTiACH5 and pTiBo542). The superbinary system has proven to be particularly useful in transformation of monocot plant species. See Hiei et al, (1994) Plant J. (6:271-282 and Ishida et al, (1996) Nat. Biotechnol. 14:745-750.

It will be clear that T-DNA vectors can also be prepared by conventional cloning techniques, as described herein after, instead of via the above described binary homologous recombination system.

According to the present invention, a chimeric gene encoding an RGEN comprises a plant-expressible promoter (preferably a DNA polymerase II “pol II” promoter), such as the promoters described above, operably linked to a DNA region encoding the RGEN and optionally a 3′end region functional in plant cells. Further elements can be operably linked in the chimeric gene to optimize expression of the RGEN. Further elements, such as enhancers or introns, can be operably linked in the chimeric gene to optimize expression of the RGEN. The chimeric gene may also comprise, in combination with the promoter, other regulatory sequences, which are located between the promoter and the coding sequence, such as transcription activators (“enhancers”), for instance the translation activator of the tobacco mosaic virus (TMV) described in Application WO 87/07644, or of the tobacco etch virus (TEV) described by Carrington & Freed 1990, J. Virol. 64: 1590-1597, for example.

Examples of introns which may be incorporated include the 5′ introns from the rice actin 1 gene (see U.S. Pat. No. 5,641,876), the rice actin 2 gene, the maize sucrose synthase gene (Clancy and Hannah, 2002, Plant Physiol. 130(2):918-29), the maize alcohol dehydrogenase-1 (Adh-1) and Bronze-1 genes (Callis et al. 1987 Genes Dev. 1(10):1183-200; Mascarenhas et al. 1990, Plant Mol Biol. 15(6):913-20), the maize heat shock protein 70 gene (see U.S. Pat. No. 5,593,874), the maize shrunken 1 gene, the light sensitive 1 gene of Solanum tuberosum, and the heat shock protein 70 gene of Petunia hybrida (see U.S. Pat. No. 5,659,122), the replacement histone H3 gene from alfalfa (Keleman et al. 2002 Transgenic Res. 11(1):69-72) and either replacement histone H3 (histone H3.3-like) gene of Arabidopsis thaliana (Chaubet-Gigot et al., 2001, Plant Mol Biol. 45(1):17-30).

Other suitable regulatory sequences include 5′ UTRs. As used herein, a 5′UTR, also referred to as leader sequence, is a particular region of a messenger RNA (mRNA) located between the transcription start site and the start codon of the coding region. It is involved in mRNA stability and translation efficiency. For example, the 5′ untranslated leader of a petunia chlorophyll a/b binding protein gene downstream of the 35S transcription start site can be utilized to augment steady-state levels of reporter gene expression (Harpster et al., 1988, Mol Gen Genet. 212(1):182-90). WO95/006742 describes the use of 5′ non-translated leader sequences derived from genes coding for heat shock proteins to increase transgene expression.

The chimeric gene may also comprise a 3′ end region, i.e. a transcription termination or polyadenylation sequence, operable in plant cells. As a transcription termination or polyadenylation sequence, use may be made of any corresponding sequence of bacterial origin, such as for example the nos terminator of Agrobacterium tumefaciens, of viral origin, such as for example the CaMV 35S terminator, or of plant origin, such as for example a histone terminator as described in published Patent Application EP 0 633 317 A1. The polyadenylation region can be derived from the natural gene, from a variety of other plant genes, or from T-DNA. The 3′ end sequence to be added may be derived from, for example, the nopaline synthase or octopine synthase genes, or alternatively from another plant gene.

According to the present invention, a chimeric gene encoding at least one guide polynucleotide comprises a plant-expressible promoter (a DNA polymerase III “pol III” promoter) operably linked to a DNA region encoding the guide polynucleotide (gRNA). Further elements, such as enhancers or introns, can be operably linked in the chimeric gene to optimize expression of the guide polynucleotide.

The chimeric gene encoding said at least one guide polynucleotide can also encode two or more guide polynucleotide sequences (gRNAs) linked by cleavage sequences, so as to enable multiplexing, i.e. targeting multiple DNA sequences simultaneously. This has e.g. been described in WO2015099850 (Csy4 cleavage sites), WO20160614811 (tRNA cleavage sequences) and WO2014204724.

In one embodiment, said chimeric gene encoding said RGEN, said at least one chimeric gene encoding said at least one guide polynucleotide and said at least one donor polynucleotide are located on one T-DNA vector. Such a T-DNA vector can comprise one or more T-DNA molecules together harbouring the (at least) three components, i.e. the chimeric gene encoding said RGEN, the at least one chimeric gene encoding said at least one guide polynucleotide and the donor polynucleotide. For example, all (at least) three components can be located on separate T-DNAs in said one T-DNA vector, each component being flanked by a pair of T-DNA borders (left and right). In another example, the chimeric gene encoding said RGEN and the at least one chimeric gene encoding said at least one guide polynucleotide could be located together on one T-DNA molecule (between one set of T-DNA borders, left and right) and the guide polynucleotide on another T-DNA molecule (between another set of T-DNA borders, left and right). In a particular example, said chimeric gene encoding said RGEN, said at least one chimeric gene encoding said at least one guide polynucleotide and said at least one donor polynucleotide are located together on one T-DNA molecule, i.e. all are located between a single set of T-DNA borders (a left and a right border).

In a particular embodiment, the coding region of the RGEN is optimized for expression in plants. It can also be optimized for expression in a particulate plant species, e.g. rice or wheat. Plant-optimized coding regions for RGENs including Cas9 have been described inter alia in Shan et al. Nature Protocols, 9, 2395-2410; WO2015026883; WO2015026885; WO2015026886.

In one embodiment, wherein said chimeric gene encoding said RGEN comprises the nucleotide sequence of SEQ ID NO. 5 from nucleotide position 28 to nucleotide position 4164.

In one embodiment, the RGEN can comprise an amino acid sequence having at least 75%, at least 80%, at least 85%, at least 90%, at least 95%, at least 98% or 100% sequence identity to aa 10-1388 of SEQ ID NO. 6 and comprising a D to E substitution at the amino acid position corresponding to position 24 of SEQ ID NO. 6.

It is preferred that the RGEN comprises at least one, for example two nuclear localization signal (NLS). The NLS can in principle be located anywhere in the polypeptide, as long as it does not interfere with the functionality of the RGEN, but is preferable located at or near the N-terminus and/or C-terminus.

In a further embodiment of the methods according to the invention, the bacterium further comprises a selectable or screenable marker gene that is introduced into and expressed in said plant cell. “Selectable or screenable markers” as used herein have their usual meaning in the art and include, but are not limited to plant expressible phosphinotricin acetyltransferase, neomycine phosphotransferase, glyphosate oxidase, glyphosate tolerant EPSP enzyme, nitrilase gene, mutant acetolactate synthase or acetohydroxyacid synthase gene, 3-glucoronidase (GUS), R-locus genes, green ditfluorescent protein and the likes. A selectable or screenable marker gene, when expressed in a plant cell or plant, can confer to said plant cell or plant a selectable or screenable phenotype. Said selectable marker gene can be on a separate T-DNA molecule or be combined with one or more or all of the other (at least) three components on one T-DNA.

Such a co-delivery of the selectable or screenable marker gene with the other components using one bacterium (e.g. on one T-DNA vector or even on one T-DNA) greatly increases the recovery of events in which the intended modification is made. It has presently been found that when selecting on the co-introduced selectable marker (i.e as a first selection) almost half of the events displaying tolerance conferred by the selectable marker gene indeed contained the desired modification.

In another embodiment, the chimeric gene encoding the RGEN, the at least one chimeric gene encoding said at least one guide polynucleotide and the at least one donor polynucleotide are delivered using one bacterium, as described herein, while the selectable or screenable marker gene is introduced into the plant cell separately, e.g. by co-cultivation with a separate bacterium comprising said selectable marker gene or another delivery technique (e.g. direct delivery).

Alternatively (or additionally), the modification that is made in the genome of the plant cell upon incorporation of the polynucleotide of interest confers upon said plant cell a selectable or screenable phenotype.

A selectable or screenable phenotype, as used herein is a characteristic conferred upon a plant cell or plant that that allows the discrimination and/or singling out and/or enrichment of said plant cell or plant from other plant cells or plants not having said characteristic. This can e.g. be a visual marker (e.g. a colour or fluorescent marker) or a selective advantage under certain conditions, such as selective agents (e.g. herbicides, antibiotics).

Conveniently, the selectable or screenable phenotype can be herbicide tolerance, such as tolerance to EPSPS inhibitor herbicides, e.g. glyphosate. To this end, the donor polynucleotide and the polynucleotide of interest can be designed such as to introduce mutations into the native EPSPS gene present in the genome of the plant cell that increase tolerance to herbicides such as glyphosate. A particular example is TIPS mutation, as e.g. described in Li et al., 2016, Nature Plants. Likewise, the donor polynucleotide can be designed to modify other plant endogenous genes that allow selection upon modification. For example, endogenous genes such as ALS/AHAS, ACCase, HPPD can be modified to modulate (increase) tolerance to the corresponding herbicides (which are well known in the art). Alternatively, a complete coding sequence of a selectable marker gene can be introduced at a specific genomic locus, whereby it is placed under the control of the required regulatory elements (such as promoters, terminators) by either choosing the genomic location so as to employ existing regulatory elements, e.g. by replacing the coding sequence of an existing gene (which can be an endogenous gene but also a transgene) by the coding sequence of the selectable or screenable marker gene, or by introducing an entire gene including regulatory sequences as well as the coding sequence.

The selectable or screenable phenotype can e.g. be (increased) tolerance to glyphosate in case of a modified EPSPS gene, can e.g. be (increased) tolerance to imidazolinones, pyrimidinylthiobenzoates, sulfonylaminocarbonyltriazolinones, sulfonylureas and/or triazolopyrimidines in case of a modified ALS/AHAS gene, can e.g. be (increased) tolerance to Aryloxyphenoxypropionate (FOPs), cyclohexanedione (DIMs), and phenylpyrazolin (DENs) in case of a modified ACCase gene, can e.g. be (increased) tolerance to pyrazolones, triketones, and diketonitriles (for example mesotrione, isoxaflutole, topramezone, pyrasulfutole and tembotrione) in case of a modified HPPD gene, etc.

In another embodiment, the selectable phenotype conferred to said plant cell by the targeted modification can be used for direct selection on the selection compound to which tolerance in conferred by the targeted genomic modification, i.e. without requiring a first selection based on a co-transformed selectable marker gene (e.g. the bar gene). This allows screening for the targeted modification in a very early stage and reduces the need for a second selection or screening step to confirm the presence of the intended modification.

Transformation of plant cells using Agrobacterium or any other bacteria can occur via protoplast co-cultivation, explant inoculation, floral dipping and vacuum infiltration. Such technologies are described, for example, in U.S. Pat. Nos. 5,177,010, 5,104,310, European Patent Application No. 0131624B1, European Patent Application No. 120516, European Patent Application No. 159418B1, European Patent Application No. 176112, U.S. Pat. Nos. 5,149,645, 5,469,976, 5,464,763, 4,940,838, 4,693,976, European Patent Application No. 116718, European Patent Application No. 290799, European Patent Application No. 320500, European Patent Application No. 604662, European Patent Application No. 627752, European Patent Application No. 0267159, European Patent Application No. 0292435, U.S. Pat. Nos. 5,231,019, 5,463,174, 4,762,785, 5,004,863, and 5,159,135. The use of T-DNA-containing vectors for the transformation of plant cells has been intensively researched and sufficiently described in European Patent Application 120516; An et al, (1985, EMBO J. 4:277-284), Fraley et al, (1986, Crit. Rev. Plant Sci. 4: 1-46), and Lee and Gelvin (2008, Plant Physiol. 146: 325-332).

Various tissue explants that can be transformed according to the invention include explants from hypocotyl, cotyledon, immature zygotic embryos, leaves, anthers, petals, ovules, roots, and meristems, stem cells and petioles. Also callus tissue can be transformed according to the invention. The term “callus”, as used herein, refers to a disorganized mass of mainly embryogenic cells and cell clusters produced as a consequence of plant tissue culture. Friable callus refers to callus with a friable texture with the potential to form shoots and roots and eventually regenerate into whole plants. Compact callus can also have the potential to form shoots and roots. Callus can be regenerated/induced from various tissue explants as mentioned above.

In one embodiment, the plant cell of which the genome is modified according to the invention is comprised within an immature embryo or embryogenic callus, i.e. the cell is a cell of an immature embryo (an immature embryo cell) or of embryogenic callus (an embryogenic callus cell), as described below.

To guide the incorporation of the polynucleotide of interest, the donor DNA molecule may comprises one or two homology regions having sufficient length and sequence identity to the genomic DNA upstream and/or downstream of the preselected site to allow recombination with the upstream and/or downstream DNA regions flanking the preselected site. This allows to better control the insertion of DNA of interest. Indeed, integration by homologous recombination will allow precise joining of the DNA of interest to the plant nuclear genome up to the nucleotide level.

To have sufficient homology for recombination, the homology region(s) may vary in length, and should be at least about 10 nucleotides in length. However, the flanking region may be as long as is practically possible (e.g. up to about 100-150 kb such as complete bacterial artificial chromosomes (BACs). Preferably, the flanking region will be about 10 nt, 15 nt, 20 nt, 25 nt, 50 nt, 100 nt, 200 nt, 500 nt, 750 nt, 1000 nt, 1500 nt, 2000 nt, 2500 nt, 5000 nt, or even longer. Moreover, the homology region(s) need(s) not be identical to the DNA region(s) flanking the preselected site) and may have between about 80% to about 100% sequence identity, preferably about 95% to about 100% sequence identity with the DNA regions flanking the preselected site. The longer the flanking region, the less stringent the requirement for homology. Furthermore, it is preferred that the sequence identity is as high as practically possible in the vicinity of the DSB. Furthermore, to achieve exchange of the target DNA sequence at the preselected site without changing the DNA sequence of the adjacent DNA sequences, the flanking DNA sequences should preferably be identical to the upstream and downstream DNA regions flanking the preselected site or the target DNA sequence to be exchanged.

The donor polynucleotide (polynucleotide of interest) may comprises one or more plant-expressible gene(s) of interest or part of one or more plant expressible genes. Such a plant expressible gene of interest can for example be a herbicide tolerance gene, an insect resistance gene, a disease resistance gene, an abiotic stress resistance gene, an enzyme involved in oil biosynthesis, carbohydrate biosynthesis, an enzyme involved in fiber strength or fiber length, an enzyme involved in biosynthesis of secondary metabolites, as are further described below.

The donor polynucleotide (polynucleotide of interest) may also comprise a selectable or screenable marker, which may or may not be removed after insertion, e.g as described in WO 06/105946, WO08/037436 or WO08/148559, to facilitate the identification of potentially correctly targeted events. This selectable or screenable marker gene preferably is different from any other marker gene that may otherwise be transferred into the plant cell.

It will be clear that also more than one Agrobacterium strain can be delivered simultaneously for multiplexing, see Char et al., (Plant Biotechnology Journal, 5 Sep. 2016).

In a further step, the thus generated and selected plant cell comprising the targeted modification may be grown into a plant. Such a plant comprising the targeted modification can subsequently be crossed with another plant. Progeny plants thereof can then be selected that comprise the intended modification, but for instance do not comprise said chimeric gene encoding said RGEN and/or said at least one chimeric gene encoding said at least one guide polynucleotide and/or non-targeted insertions of the donor polynucleotide. Crossing with another plant can also be selfing. Such a plant comprising the targeted modification can also be used to produce a plant product, as described elsewhere herein.

It will be appreciated that the methods of this aspect of invention can be applied to any plant cell or plant amenable to bacterial transformation. In one example, the plant cell or plant is a rice species (Oryza), e.g. Oryza sativa.

It is also an object of the invention to provide plant cells, plant parts and plants generated according to the methods of the invention, such as fruits, seeds, embryos, reproductive tissue, meristematic regions, callus tissue, leaves, roots, shoots, flowers, fibers, vascular tissue, gametophytes, sporophytes, pollen and microspores, which are characterised in that they comprise the intended modification in the (nuclear) genome (insertion, replacement and/or deletion). Gametes, seeds, embryos, either zygotic or somatic, progeny or hybrids of plants comprising the DNA modification events, which are produced by traditional breeding methods, are also included within the scope of the present invention. Such plants may contain the polynucleotide of interest inserted at or replacing the preselected site or may have a specific DNA sequence deleted (even single nucleotides), and will only be different from their progenitor plants by the presence of this intended modification.

In a second aspect, the invention provides a bacterium suitable for use in the above methods. Such a bacterium comprises a chimeric gene encoding an RGEN, at least one chimeric gene encoding at least one guide polynucleotide and at least one donor polynucleotide, wherein said bacterium is capable of transferring said chimeric gene encoding said RGEN, said chimeric gene encoding said guide polynucleotide and said donor polynucleotide into (the nuclear genome of) a plant cell, wherein said RGEN and said guide polynucleotide upon expression in said plant cell are capable of forming a complex that enables the RGEN to introduce a DNA break at a preselected site in the (nuclear) genome of a plant cell and wherein said donor polynucleotide is to be used as a template for repair of said DNA break, all as described in any of the embodiments of the first aspect above.

Also provided is a (T-DNA) vector comprising the chimeric gene encoding an RGEN, the at least one chimeric gene encoding at least one guide polynucleotide and the at least one donor polynucleotide as described herein. In a further embodiment, said vector also comprises a screenable or selectable marker gene as described herein. Preferably, said components are located on one T-DNA molecule (between a pair of T-DNA borders).

In a third aspect, the invention provided an isolated RGEN polypeptide as described in the above aspect, such as a Cas9 polypeptide, comprising a D to E substitution at the amino acid position corresponding to position 24 of SEQ ID NO. 6. In one example, the isolated RGEN polypeptide has at least 75%, at least 80%, at least 85%, at least 90%, at least 95%, at least 98% or 100% sequence identity to SEQ ID NO 6 from amino acid position 10 to amino acid position 1388.

Also included within the scope of the present invention is an isolated nucleic acid encoding the RGEN as described above, for example wherein said nucleic acid comprised the nucleotide sequence of SEQ ID NO. 5 from nucleotide position 28 to nucleotide position 4164 or variants thereof having 1, 2, 3, 4, 5, 6, 7, 8, 9, 10, 15, 20, 25 nt differences with respect to SEQ ID 5, while encoding the isolated RGEN polypeptide as above.

In another embodiment, a chimeric gene is provided comprising the isolated nucleic acid as described above operably linked to a heterologous promoter.

Further provided is a host cell, such as a bacterial cell or a plant cell, comprising the isolated polypeptide, the isolated nucleic acid or the chimeric gene as described above.

In a fourth aspect, a method is provided for modifying an endogenous EPSPS gene in a plant cell or for producing a plant cell having a modified EPSPS gene, comprising the steps of:
a. expressing in said cell a site-directed DNA modifying polypeptide recognising a sequence in an endogenous EPSPS gene of said plant and/or introducing into said plant cell a donor polynucleotide that can be used as a template for modifying said endogenous EPSPS gene;
b. evaluating tolerance of said plant cell to one or more EPSPS inhibitors by culturing said plant cell on medium comprising said EPSPS inhibitor or inhibitors; and optionally
c. selecting a plant cell having increased tolerance to said at least one EPSPS inhibitor (compared to said plant cell prior to the modification).

The direct selection on EPSPS inhibitors (e.g. glyphosate), i.e. an EPSPS inhibitors is used as a first selection agent, allows an easy readout for the efficiency etc of the method. Thus, this method can conveniently be used for evaluating genome modification components (genome editing components), such as donor polynucleotides, guide polynucleotides, site specific nucleases, e.g. meganucleases, zinc finger nucleases (ZFNs), TAL-effector nucleases (TALENs), RGENs, DNA guided nucleases or other spite-directed or sequence specific DNA modifying enzymes that can introduce mutations (e.g. deaminase), as well as elements used for the expression of such components such as promoters, as well as of other parameters that can affect the outcome. e.g. in terms of efficiency, purity of events, such as delivery methods/machines, e.g. particle bombardment, bacterial transformation, (ribonucleo)protein transfection, timing of delivery of the various components etc. Also, by selecting a plant cell having increased tolerance a cell can be selected having a modified EPSPS gene. Thus, this method also allows the production of a plant cell having a modified EPSPS gene, e.g. a plant cell having modified (e.g. increased or decreased) tolerance to EPSPS inhibitor herbicides.

In one embodiment, the selection of plant cells having a modified EPSPS gene, i.e. the culturing on medium comprising EPSPS inhibitors, takes place a few days (e.g. 1, 2, 3, 4 or 5 days) after first culturing the cells on a non-selective medium directly after transformation. The EPSPS inhibitor can be glyphosate. The medium can comprise glyphosate in a concentration of about 50-250 mg/L, such as about 100-200 mg/L, such as about 150 mg/L.

In one embodiment, the donor polynucleotide comprises the TIPS mutation, i.e. when used as a template for modifying said endogenous EPSPS gene results in introduction of the TIPS mutation into said EPSPS gene. In alternative embodiments, the donor polynucleotide may comprise the TIPV or TIPL mutation.

In another embodiment, the plant cell is a rice plant cell.

In a further embodiment, no (functional) selectable marker gene is introduced into the plant cell, i.e. the method excludes the introduction of a separate (functional) selectable marker gene.

Expressing a site-directed DNA modifying polypeptide in a plant cell can conveniently be achieved by providing the plant cell with a plant-expressible gene encoding the polypeptide, according to any method available in the art, such as agrobacterium-mediated transformation, direct delivery methods such as bombardment or viral delivery and the like. Alternatively, the plant cell can be directly provided with the polypeptide, optionally in conjunction with a guide polynucleotide, as is described in the art (see e.g. WO2014065596).

In a fifth aspect, the invention provides a method for modifying the (nuclear) genome of a plant cell at a preselected site or for producing a plant cell having a modification at a preselected site in the (nuclear) genome), comprising the steps of:
a. Introducing/Expressing in said cell an a nucleotide-guided DNA modifying polypeptide (NGDMP) and a guide polynucleotide, wherein said NGDMP and said guide polynucleotide are capable of forming a complex that enables the NGDMP to modify the genome of a plant cell at a preselected site;
b. Selecting a plant cell wherein said genome has been modified at said preselected site characterised in that said NGDMP, said guide polynucleotide are introduced to said plant cell using a particle inflow gun.

A particle inflow gun, as used herein, refers to a device allowing acceleration of DNA coated gold particles directly in a helium steam, as described e.g. by Vain, P, Keen, N. Murillo, J. et al. Plant Cell Tiss Organ Cult (1993) 33: 237.

In another aspect, a method is provided for modifying the (nuclear) genome of a plant cell at a preselected site, or for producing a plant cell with a modified genome, comprising the steps of:
a. introducing into said cell a nucleotide-guided DNA modifying polypeptide (NGDMP) and a guide polynucleotide, wherein said NGDMP and guide polynucleotide are capable of forming a complex that enables the NGDMP to modify the genome of a plant cell at a preselected site;
b. introducing into said cell at least one (plant-expressible) selectable marker gene;
c. selecting one or more plant cells comprising said selectable marker gene (i.e. selecting one or more plant cells having the selectable phenotype conferred by said selectable marker gene);
d. selecting (from said one or more plants cells) a plant cell wherein said genome has been modified at said preselected site
characterised in that said NGDMP, said at least one guide polynucleotide and said at least one selectable marker gene are introduced into said plant cell by contacting said plant cell with at least one bacterium comprising a chimeric gene encoding said RGEN, at least one chimeric gene encoding said at least one guide polynucleotide and at least one polynucleotide comprising said selectable marker gene.

A nucleotide-guided DNA modifying polypeptide (NGDMP) can be a nucleotide-guided endonuclease, e.g. an RGEN as described above, or a DNA-guided endonuclease (e.g. WO2014189628; WO2015140347; Nature Biotechnology 34, 768-773, 2016), or other nucleotide-guided (e.g. RNA-guided or DNA-guided) DNA modifying polypeptide, such as epigenetic modifiers (e.g. methylases), deaminases (base editing), as e.g. described in WO2013176772, WO2013088446, WO2014099750.

Accordingly, “modifying” or “modified” as used herein, can refer to a change in the nucleotide sequence at the preselected site, e.g. due to cleavage and subsequent repair or by base editing. It can also refer to a change in the epigenetic state, e.g. DNA methylation, chromatin structure, histone modifications at or around the preselected site, that can influence for example expression of a nearby gene.

Thus, in one embodiment, said RGDMP is an RGEN, said RGEN and said at least one guide polynucleotide being capable of forming a complex that enables the RGEN to introduce a DNA break at or near said preselected site.

Together with said RGEN and said guide polynucleotide a donor polynucleotide comprising a polynucleotide of interest can be introduced into said plant cell, wherein said donor polynucleotide is used as a template for repair of said DNA break, thereby integrating said polynucleotide of interest at said preselected site and resulting in a modification of said genome at said preselected site.

The invention further provides a bacterium comprising a chimeric gene encoding an NGDMP, at least one chimeric gene encoding at least one guide polynucleotide and at least one (plant-expressible) selectable marker gene, wherein said bacterium is capable of transferring or introducing said chimeric gene encoding said NGDMP, said chimeric gene encoding said guide polynucleotide and said selectable marker gene into (the nuclear genome of) a plant cell, wherein said NGDMP and said guide polynucleotide upon expression in said plant cell are capable of forming a complex that enables the NGDMP to modify the (nuclear) genome of a plant cell.

The bacterium can be any bacterium that is capable of directing the transfer of DNA contained within the bacterium stably into the genome of a plant cell, as described above. Particularly suitable is Agrobacterium tumefaciens.

In one example, the chimeric gene encoding the NGDMP, the chimeric gene encoding the guide polynucleotide and the selectable marker gene are located on one vector, preferably on one T-DNA molecule (between a pair of T-DNA borders).

The bacterium may further comprise a donor polynucleotide as described herein, e.g. for repair of the DNA break induced by an RGEN. In a preferred embodiment, the donor polynucleotide is located on the same T-DNA.

Also described is a (T-DNA) vector comprising the chimeric gene encoding an NGDMP, the chimeric gene encoding a guide polynucleotide and the selectable marker gene as described herein, preferably on one T-DNA molecule (between a pair of T-DNA borders). The vector can also comprise a donor nucleotide, preferably on the same T-DNA.

It will be clear that via the donor polynucleotide the methods according to the invention allow insertion of any nucleic acid molecule of interest including nucleic acid molecule comprising genes encoding an expression product (genes of interest), nucleic acid molecules comprising a nucleotide sequence with a particular nucleotide sequence signature e.g. for subsequent identification, or nucleic acid molecules comprising or modifying (inducible) enhancers or silencers, e.g. to modulate the expression of genes located near the preselected site.

Herbicide-tolerance genes include a gene encoding the enzyme 5-enolpyruvylshikimate-3-phosphate synthase (EPSPS). Examples of such EPSPS genes are the AroA gene (mutant CT7) of the bacterium Salmonella typhimurium (Comai et al., 1983, Science 221, 370-371), the CP4 gene of the bacterium Agrobacterium sp. (Barry et al., 1992, Curr. Topics Plant Physiol. 7, 139-145), the genes encoding a Petunia EPSPS (Shah et al., 1986, Science 233, 478-481), a Tomato EPSPS (Gasser et al., 1988, J. Biol. Chem. 263, 4280-4289), or an Eleusine EPSPS (WO 01/66704). It can also be a mutated EPSPS as described in for example EP 0837944, WO 00/66746, WO 00/66747 or WO02/26995. Glyphosate-tolerant plants can also be obtained by expressing a gene that encodes a glyphosate oxido-reductase enzyme as described in U.S. Pat. Nos. 5,776,760 and 5,463,175. Glyphosate-tolerant plants can also be obtained by expressing a gene that encodes a glyphosate acetyl transferase enzyme as described in for example WO 02/36782, WO 03/092360, WO 05/012515 and WO 07/024782. Glyphosate-tolerant plants can also be obtained by selecting plants containing naturally-occurring mutations of the above-mentioned genes, as described in for example WO 01/024615 or WO 03/013226. EPSPS genes that confer glyphosate tolerance are described in e.g. U.S. patent application Ser. Nos. 11/517,991, 10/739,610, 12/139,408, 12/352,532, 11/312,866, 11/315,678, 12/421,292, 11/400,598, 11/651,752, 11/681,285, 11/605,824, 12/468,205, 11/760,570, 11/762,526, 11/769,327, 11/769,255, 11/943,801 or 12/362,774. Other genes that confer glyphosate tolerance, such as decarboxylase genes, are described in e.g. US patent application Ser. Nos. 11/588,811, 11/185,342, 12/364,724, 11/185,560 or 12/423,926.

Other herbicide tolerance genes may encode an enzyme detoxifying the herbicide or a mutant glutamine synthase enzyme that is resistant to inhibition, e.g. described in U.S. patent application Ser. No. 11/760,602. One such efficient detoxifying enzyme is an enzyme encoding a phosphinothricin acetyltransferase (such as the bar or pat protein from Streptomyces species). Phosphinothricin acetyltransferases are for example described in U.S. Pat. Nos. 5,561,236; 5,648,477; 5,646,024; 5,273,894; 5,637,489; 5,276,268; 5,739,082; 5,908,810 and 7,112,665.

Herbicide-tolerance genes may also confer tolerance to the herbicides inhibiting the enzyme hydroxyphenylpyruvatedioxygenase (HPPD). Hydroxyphenylpyruvatedioxygenases are enzymes that catalyze the reaction in which para-hydroxyphenylpyruvate (HPP) is transformed into homogentisate. Plants tolerant to HPPD-inhibitors can be transformed with a gene encoding a naturally-occurring resistant HPPD enzyme, or a gene encoding a mutated or chimeric HPPD enzyme as described in WO 96/38567, WO 99/24585, and WO 99/24586, WO 2009/144079, WO 2002/046387, or U.S. Pat. No. 6,768,044. Tolerance to HPPD-inhibitors can also be obtained by transforming plants with genes encoding certain enzymes enabling the formation of homogentisate despite the inhibition of the native HPPD enzyme by the HPPD-inhibitor. Such plants and genes are described in WO 99/34008 and WO 02/36787. Tolerance of plants to HPPD inhibitors can also be improved by transforming plants with a gene encoding an enzyme having prephenate deshydrogenase (PDH) activity in addition to a gene encoding an HPPD-tolerant enzyme, as described in WO 2004/024928. Further, plants can be made more tolerant to HPPD-inhibitor herbicides by adding into their genome a gene encoding an enzyme capable of metabolizing or degrading HPPD inhibitors, such as the CYP450 enzymes shown in WO 2007/103567 and WO 2008/150473.

Still further herbicide tolerance genes encode variant ALS enzymes (also known as acetohydroxyacid synthase, AHAS) as described for example in Tranel and Wright (2002, Weed Science 50:700-712), but also, in U.S. Pat. Nos. 5,605,011, 5,378,824, 5,141,870, and 5,013,659. The production of sulfonylurea-tolerant plants and imidazolinone-tolerant plants is described in U.S. Pat. Nos. 5,605,011; 5,013,659; 5,141,870; 5,767,361; 5,731,180; 5,304,732; 4,761,373; 5,331,107; 5,928,937; and 5,378,824; and international publication WO 96/33270. Other imidazolinone-tolerance genes are also described in for example WO 2004/040012, WO 2004/106529, WO 2005/020673, WO 2005/093093, WO 2006/007373, WO 2006/015376, WO 2006/024351, and WO 2006/060634. Further sulfonylurea- and imidazolinone-tolerance genes are described in for example WO 07/024782.

Insect resistance gene may comprise a coding sequence encoding:

1) an insecticidal crystal protein from Bacillus thuringiensis or an insecticidal portion thereof, such as the insecticidal crystal proteins listed by Crickmore et al. (1998, Microbiology and Molecular Biology Reviews, 62: 807-813), updated by Crickmore et al. (2005) at the Bacillus thuringiensis toxin nomenclature, online at:

http://www.lifesci.sussex.ac.uk/Home/Neil_Crickmore/Bt/), or insecticidal portions thereof, e.g., proteins of the Cry protein classes Cry1Ab, Cry1Ac, Cry1B, Cry1C, Cry1D, Cry1F, Cry2Ab, Cry3Aa, or Cry3Bb or insecticidal portions thereof (e.g. EP 1999141 and WO 2007/107302), or such proteins encoded by synthetic genes as e.g. described in and U.S. patent application Ser. No. 12/249,016; or

2) a crystal protein from Bacillus thuringiensis or a portion thereof which is insecticidal in the presence of a second other crystal protein from Bacillus thuringiensis or a portion thereof, such as the binary toxin made up of the Cry34 and Cry35 crystal proteins (Moellenbeck et al. 2001, Nat. Biotechnol. 19: 668-72; Schnepf et al. 2006, Applied Environm. Microbiol. 71, 1765-1774) or the binary toxin made up of the Cry1A or Cry1F proteins and the Cry2Aa or Cry2Ab or Cry2Ae proteins (U.S. patent application Ser. No. 12/214,022); or

3) a hybrid insecticidal protein comprising parts of different insecticidal crystal proteins from Bacillus thuringiensis, such as a hybrid of the proteins of 1) above or a hybrid of the proteins of 2) above, e.g., the Cry1A.105 protein produced by corn event MON89034 (WO 2007/027777); or

4) a protein of any one of 1) to 3) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation, such as the Cry3Bb1 protein in corn events MON863 or MON88017, or the Cry3A protein in corn event MIR604; or

5) an insecticidal secreted protein from Bacillus thuringiensis or Bacillus cereus, or an insecticidal portion thereof, such as the vegetative insecticidal (VIP) proteins listed at: http://www.lifesci.sussex.ac.uk/home/Neil_Crickmore/Bt/vip.html, e.g., proteins from the VIP3Aa protein class; or

6) a secreted protein from Bacillus thuringiensis or Bacillus cereus which is insecticidal in the presence of a second secreted protein from Bacillus thuringiensis or B. cereus, such as the binary toxin made up of the VIP1A and VIP2A proteins (WO 94/21795); or

7) a hybrid insecticidal protein comprising parts from different secreted proteins from Bacillus thuringiensis or Bacillus cereus, such as a hybrid of the proteins in 1) above or a hybrid of the proteins in 2) above; or

8) a protein of any one of 5) to 7) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation (while still encoding an insecticidal protein), such as the VIP3Aa protein in cotton event COT102; or

9) a secreted protein from Bacillus thuringiensis or Bacillus cereus which is insecticidal in the presence of a crystal protein from Bacillus thuringiensis, such as the binary toxin made up of VIP3 and Cry1A or Cry1F, or the binary toxin made up of the VIP3 protein and the Cry2Aa or Cry2Ab or Cry2Ae proteins (U.S. patent application Ser. No. 12/214,022);

10) a protein of 9) above wherein some, particularly 1 to 10, amino acids have been replaced by another amino acid to obtain a higher insecticidal activity to a target insect species, and/or to expand the range of target insect species affected, and/or because of changes introduced into the encoding DNA during cloning or transformation (while still encoding an insecticidal protein).

An “insect-resistant gene as used herein, further includes transgenes comprising a sequence producing upon expression a double-stranded RNA which upon ingestion by a plant insect pest inhibits the growth of this insect pest, as described e.g. in WO 2007/080126, WO 2006/129204, WO 2007/074405, WO 2007/080127 and WO 2007/035650.

Abiotic stress tolerance genes include

1) a transgene capable of reducing the expression and/or the activity of poly(ADP-ribose) polymerase (PARP) gene in the plant cells or plants as described in WO 00/04173, WO/2006/045633.

2) a transgene capable of reducing the expression and/or the activity of the PARG encoding genes of the plants or plants cells, as described e.g. in WO 2004/090140.

3) a transgene coding for a plant-functional enzyme of the nicotineamide adenine dinucleotide salvage synthesis pathway including nicotinamidase, nicotinate phosphoribosyltransferase, nicotinic acid mononucleotide adenyl transferase, nicotinamide adenine dinucleotide synthetase or nicotine amide phosphorybosyltransferase as described e.g. in PCT/EP07/002433, EP 1999263, or WO 2007/107326.

Enzymes involved in carbohydrate biosynthesis include those described in e.g. EP 0571427, WO 95/04826, EP 0719338, WO 96/15248, WO 96/19581, WO 96/27674, WO 97/11188, WO 97/26362, WO 97/32985, WO 97/42328, WO 97/44472, WO 97/45545, WO 98/27212, WO 98/40503, WO99/58688, WO 99/58690, WO 99/58654, WO 00/08184, WO 00/08185, WO 00/08175, WO 00/28052, WO 00/77229, WO 01/12782, WO 01/12826, WO 02/101059, WO 03/071860, WO 2004/056999, WO 2005/030942, WO 2005/030941, WO 2005/095632, WO 2005/095617, WO 2005/095619, WO 2005/095618, WO 2005/123927, WO 2006/018319, WO 2006/103107, WO 2006/108702, WO 2007/009823, WO 00/22140, WO 2006/063862, WO 2006/072603, WO 02/034923, WO 01/14569, WO 02/79410, WO 03/33540, WO 2004/078983, WO 01/19975, WO 95/26407, WO 96/34968, WO 98/20145, WO 99/12950, WO 99/66050, WO 99/53072, U.S. Pat. No. 6,734,341, WO 00/11192, WO 98/22604, WO 98/32326, WO 01/98509, WO 01/98509, WO 2005/002359, U.S. Pat. Nos. 5,824,790, 6,013,861, WO 94/04693, WO 94/09144, WO 94/11520, WO 95/35026 or WO 97/20936 or enzymes involved in the production of polyfructose, especially of the inulin and levan-type, as disclosed in EP 0663956, WO 96/01904, WO 96/21023, WO 98/39460, and WO 99/24593, the production of alpha-1,4-glucans as disclosed in WO 95/31553, US 2002031826, U.S. Pat. Nos. 6,284,479, 5,712,107, WO 97/47806, WO 97/47807, WO 97/47808 and WO 00/14249, the production of alpha-1,6 branched alpha-1,4-glucans, as disclosed in WO 00/73422, the production of alternan, as disclosed in e.g. WO 00/47727, WO 00/73422, U.S. Pat. No. 5,908,975 and EP 0728213, the production of hyaluronan, as for example disclosed in WO 2006/032538, WO 2007/039314, WO 2007/039315, WO 2007/039316, JP 2006304779, and WO 2005/012529.

Plants (Angiospermae or Gymnospermae) include for example cotton, canola, oilseed rape, soybean, vegetables, potatoes, Lemna spp., Nicotiana spp., Arabidopsis, alfalfa, barley, bean, corn, cotton, flax, millet, pea, rape, rice, rye, safflower, sorghum, soybean, sunflower, tobacco, turfgrass, wheat, asparagus, beet and sugar beet, broccoli, cabbage, carrot, cauliflower, celery, cucumber, eggplant, lettuce, onion, oilseed rape, pepper, potato, pumpkin, radish, spinach, squash, sugar cane, tomato, zucchini, almond, apple, apricot, banana, blackberry, blueberry, cacao, cherry, coconut, cranberry, date, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut and watermelon.

In one embodiment, also provided are plant cells, plant parts and plants generated according to the methods of the invention, such as fruits, seeds, embryos, reproductive tissue, meristematic regions, callus tissue, leaves, roots, shoots, flowers, fibers, vascular tissue, gametophytes, sporophytes, pollen and microspores, which are characterised in that they comprise a specific modification in the genome (insertion, replacement and/or deletion). Gametes, seeds, embryos, either zygotic or somatic, progeny or hybrids of plants comprising the DNA modification events, which are produced by traditional breeding methods, are also included within the scope of the present invention. Such plants may contain a nucleic acid molecule of interest inserted at or instead of a target sequence or may have a specific DNA sequence deleted (even single nucleotides), and will only be different from their progenitor plants by the presence of this heterologous DNA or DNA sequence or the absence of the specifically deleted sequence (i.e. the intended modification) compared to the original plant cell or plant before the modification.

In particular embodiments the plant cell described herein is a non-propagating plant cell, or a plant cell that cannot be regenerated into a plant, or a plant cell that cannot maintain its life by synthesizing carbohydrate and protein from the inorganics, such as water, carbon dioxide, and inorganic salt, through photosynthesis.

The invention further provides a method for producing a plant comprising a modification at a predefined site of the genome, comprising the step of crossing a plant generated according to the above methods with another plant or with itself and optionally harvesting seeds.

The invention further provides a method for producing feed, food or fiber comprising the steps of providing a population of plants generated according to the above methods and harvesting seeds.

The plants and seeds according to the invention may be further treated with a chemical compound, e.g. if having tolerance to such a chemical.

Accordingly, the invention also provides a method of growing a plant generated according to the above methods, comprising the step of applying a chemical to said plant or substrate wherein said plant is grown.

Further provided is a process of growing a plant in the field comprising the step of applying a chemical compound on a plant generated according to the above methods.

Also provided is a process of producing treated seed comprising the step applying a chemical compound, such as the chemicals described above, on a seed of plant generated according to the above described methods.

In a further embodiment, the plant obtained by the current methods (comprising the targeted modification) may be used to obtain a plant product. Thus, also provided is a method for producing a plant product, comprising obtaining a plant obtained by the methods described herein, or part thereof, and producing the plant product therefrom.

A plant product as used herein can be a food product (which may be a food ingredient), a feed product (which may be a feed ingredient) or industrial product, wherein the food or feed can e.g. be oil, meal, grain, starch, flour or protein and wherein the industrial product can be biofuel, fiber, industrial chemicals, a pharmaceutical or a nutraceutical. Animal feed can be harvested grain, hay, straw or silage. The plants obtained according to the invention may be used directly as animal feed, for example when growing in the field.

In case of e.g. a soybean plant, the plant product can be soybean meal, ground seeds, flour, or flakes, or soybean oil, soybean protein, lecithin, soybean milk, tofu, margarine, biodiesel, biocomposite, adhesive, solvent, lubricant, cleaner, foam, paint, ink, candle, or a soybean-oil or soybean protein-containing food or feed product.

In case of e.g a wheat plant or other cereal plant, examples of food products include flour, starch, leavened or unleavened breads, pasta, noodles, animal fodder, breakfast cereals, snack foods, cakes, malt, pastries, seitan and foods containing flour-based sauces.

In case of a fiber plant such as cotton flax, jute, hemp, ramie, sisal, manilla hemp, pineapple, coconut, the plant product may be a fiber, yarn, fabric, but can also be oil, meal, cake.

In case of a tomato plant, the product may be salads, sandwiches, tomato juice, tomato slices, tomato sauce, tomato paste, tomato soup, tomato ketchup and any other food product that comprises tomato such as pasta, pizza, salsa, and more.

Such a plant product may comprise a nucleic acid comprising the targeted modification or a part thereof, such as such product that comprises a nucleic acid that produces an amplicon diagnostic or specific for the targeted modification.

In some embodiments, nucleic acid molecules used to practice the invention, including the donor polynucleotide as well as nucleic acid molecules encoding e.g. the guide polynucleotide, nucleases, nicking enzymes or other DNA modifying polypeptides, may be introduced (either transiently or stably) into the cell by any means suitable for the intended host cell, e.g. viral delivery, bacterial delivery (e.g. Agrobacterium), polyethylene glycol (PEG) mediated transformation, electroporation, vacuum infiltration, lipofection, microinjection, biolistics, virosomes, liposomes, immunoliposomes, polycation or lipid:nucleic acid conjugates, naked DNA, artificial virions, and calcium-mediated delivery.

Transformation of a plant means introducing a nucleic acid molecule into a plant in a manner to cause stable or transient expression of the sequence. Transformation and regeneration of both monocotyledonous and dicotyledonous plant cells is now routine, and the selection of the most appropriate transformation technique will be determined by the practitioner. The choice of method will vary with the type of plant to be transformed; those skilled in the art will recognize the suitability of particular methods for given plant types. Suitable methods can include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium-mediated transformation.

Transformed plant cells can be regenerated into whole plants. Such regeneration techniques rely on manipulation of certain phytohormones in a tissue culture growth medium, typically relying on a biocide and/or herbicide marker that has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee (1987) Ann. Rev. of Plant Phys. 38:467-486. To obtain whole plants from transgenic tissues such as immature embryos, they can be grown under controlled environmental conditions in a series of media containing nutrients and hormones, a process known as tissue culture. Once whole plants are generated and produce seed, evaluation of the progeny begins.

A nucleic acid molecule can also be introduced into a plant by means of introgression. Introgression means the integration of a nucleic acid in a plant's genome by natural means, i.e. by crossing a plant comprising the chimeric gene described herein with a plant not comprising said chimeric gene. The offspring can be selected for those comprising the chimeric gene.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e. a position in an alignment where a residue is present in one sequence but not in the other, is regarded as a position with non-identical residues. The alignment of the two sequences is performed by the Needleman and Wunsch algorithm (Needleman and Wunsch 1970). The computer-assisted sequence alignment above, can be conveniently performed using standard software program such as GAP which is part of the Wisconsin Package Version 10.1 (Genetics Computer Group, Madison, Wis., USA) using the default scoring matrix with a gap creation penalty of 50 and a gap extension penalty of 3.

A chimeric gene, as used herein, refers to a gene that is made up of heterologous elements that are operably linked to enable expression of the gene, whereby that combination is not normally found in nature. As such, the term “heterologous” refers to the relationship between two or more nucleic acid or protein sequences that are derived from different sources. For example, a promoter is heterologous with respect to an operably linked nucleic acid sequence, such as a coding sequence, if such a combination is not normally found in nature. In addition, a particular sequence may be “heterologous” with respect to a cell or organism into which it is inserted (i.e. does not naturally occur in that particular cell or organism).

The expression “operably linked” means that said elements of the chimeric gene are linked to one another in such a way that their function is coordinated and allows expression of the coding sequence, i.e. they are functionally linked. By way of example, a promoter is functionally linked to another nucleotide sequence when it is capable of ensuring transcription and ultimately expression of said other nucleotide sequence. Two proteins encoding nucleotide sequences, e.g. a transit peptide encoding nucleic acid sequence and a nucleic acid sequence encoding a second protein, are functionally or operably linked to each other if they are connected in such a way that a fusion protein of first and second protein or polypeptide can be formed.

A gene, e.g. a chimeric gene, is said to be expressed when it leads to the formation of an expression product. An expression product denotes an intermediate or end product arising from the transcription and optionally translation of the nucleic acid, DNA or RNA, coding for such product, e. g. the second nucleic acid described herein. During the transcription process, a DNA sequence under control of regulatory regions, particularly the promoter, is transcribed into an RNA molecule. An RNA molecule may either itself form an expression product or be an intermediate product when it is capable of being translated into a peptide or protein. A gene is said to encode an RNA molecule as expression product when the RNA as the end product of the expression of the gene is, e. g., capable of interacting with another nucleic acid or protein. Examples of RNA expression products include inhibitory RNA such as e. g. sense RNA (co-suppression), antisense RNA, ribozymes, miRNA or siRNA, mRNA, rRNA and tRNA. A gene is said to encode a protein as expression product when the end product of the expression of the gene is a protein or peptide.

A plant-expressible chimeric gene is a chimeric gene capable of expression in a plant (cell). Such a chimeric gene contains a plant expressible promoter and optionally a 3′ end region functional in plant cells.

Further operably linked elements (e.g. enhancers, introns) can be included into the chimeric genes according to the invention to enhance expression of the operably linked coding sequence.

A nucleic acid or nucleotide, as used herein, refers to both DNA and RNA. DNA also includes cDNA and genomic DNA. A nucleic acid molecules can be single- or double-stranded, and can be synthesized chemically or produced by biological expression in vitro or even in vivo.

It will be clear that whenever nucleotide sequences of RNA molecules are defined by reference to nucleotide sequence of corresponding DNA molecules, the thymine (T) in the nucleotide sequence should be replaced by uracil (U). Whether reference is made to RNA or DNA molecules will be clear from the context of the application.

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a DNA region which is functionally or structurally defined may comprise additional DNA regions etc.

Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

All patents, patent applications, and publications or public disclosures (including publications on internet) referred to or cited herein are incorporated by reference in their entirety.

The sequence listing contained in the file named “BCS16-2019_ST25.txt”, which is 61 kilobytes (size as measured in Microsoft Windows®), contains 6 sequences SEQ ID NO: 1 through SEQ ID NO: 6, is filed herewith by electronic submission and is incorporated by reference herein.

The invention will be further described with reference to the examples described herein; however, it is to be understood that the invention is not limited to such examples.

### SEQUENCE LISTING

Throughout the description and Examples, reference is made to the following sequences:

SEQ ID NO. 1: Nucleotide sequence of Cas9 vector pKVA790/pBay00201

SEQ ID NO. 2: Nucleotide sequence of gRNA vector pKVA766

SEQ ID NO. 3: Nucleotide sequence of TIPS repair vector pKVA761

SEQ ID NO. 4: Nucleotide sequence of T-DNA vector pBay00461

SEQ ID NO. 5: coding sequence of plant optimized Cas9 as present in pKVA790/pBay00201 and pBay00461

SEQ ID NO. 6: amino acid sequence of plant codon-optimized Cas9 of SEQ ID NO. 5

### EXAMPLES

### Example 1: Vector Construction

Using standard molecular biology techniques, the following vectors were created, containing the following operably linked elements:

RGEN (Cas9) Expression Vector pKVA790 (Seq ID No: 1):
pubiZm (nt 431-2427): sequence including the promoter region of the ubiquitin-1 gene of Zea mays (corn) (Christensen et al., 1992).
   5′ UTR (nt 1261-2427): sequence including the leader sequence of the ubiquitin-1 gene of Zea mays (corn) (Christensen et al., 1992); contains an intron.
   Intron (nt 1481-2427): Sequence containing the first intron of the ubiquitin-1 gene of Zea mays (corn) (Christensen et al., 1992)
Cas9Sp-3Pb (nt 2430-6635): coding sequence (CDS) of a modified endonuclease CAS9 gene of Streptococcus pyogenes (Li et al., 2013), comprising at amino acid position 24 a E instead of D, further adapted to rice or wheat codon usage.
   NLSsv40 (nt 2439-2456): nuclear localization signal derived from the large T-antigen gene of simian virus 40 (Kalderon et al., 1984)
   NLSnupIXI (nt 6594-6632): nuclear localization signal of the nucleoplasmin gene of Xenopus laevis (Dingwall et al., 2187)
3′nos-N3 (nt 6646-6904): sequence including the 3′ untranslated region of the nopaline synthase gene from the T-DNA of pTiT37 (Depicker et al., 1982).

Guide RNA Expression Vector pKVA766 (SEQ ID NO: 2):
P-u6-3.1 (nt 534-1049-complement): The Pol III promoter region of the U6 gene of Oryza sativa (Jiang W. et al., 2013).
sgR-1.22 (nt 429-533-complement): Sequence encoding a synthetic guide RNA for endonuclease CAS9-mediated DNA cleavage (Li et al., 2013), targeting epsps gene of Oryza sativa.
   SiteTS3 (nt 429-448 complement): sgRNA targeting sequence
   sgR22 (nt 429-533 complement): sequence encoding a synthetic guide RNA for endonuclease CAS9-mediated DNA cleavage (Li et al., 2013) targeting the Oryza sativa EPSPS gene
   sgRNA (nt 449-525 complement): guide RNA scaffolding sequence
   TpolII (nt 526-533 complement): RNA Polymerase III termination signal

Repair DNA Vector pKVA761 for TIPS Mutation (SEQ ID NO: 3):
epspsOs-2Ga-4 (nt 404-1403): Fragment of genomic coding sequence of the modified 5-enolpyruvylshikimate-3-phosphate synthase gene of Oryza sativa, encoding for modified EPSPS protein of Oryza sativa species (unpublished)
   exon (nt 717-961):
   TIPS region (nt 888-926)
   T169I (nt 909-911)
   P173S (nt 921-923)
   Exon (nt 1041-1194)

T-DNA Vector Comprising Cas9, gRNA and TIPS Repair DNA pTKVA869/pBay00461 (SEQ ID NO: 4):
RB (nt 1 to 25): right border repeat from the T-DNA of Agrobacterium tumefaciens (Zambryski, 1988).

Cas9 Chimeric Gene:
pubiZm (nt 143-2139): sequence including the promoter region of the ubiquitin-1 gene of Zea mays (corn) (Christensen et al., 1992).
   5′ ubiZm intron1 (nt 1130-2139): Sequence containing the first intron of the ubiquitin-1 gene of Zea mays (corn) (Christensen et al., 1992)
Cas9Sp-3Pb (nt 2142-6347): coding sequence (CDS) of a modified endonuclease CAS9 gene of Streptococcus pyogenes (Li et al., 2013), adapted to rice codon usage.
   NLSsv40 (nt 2151-2168): nuclear localization signal derived from the large T-antigen gene of simian virus 40 (Kalderon et al., 1984)
   NLSnupIXI (nt 6306-6344): nuclear localization signal of the nucleoplasmin gene of Xenopus laevis (Dingwall et al., 2187)
3′nos-N3 (nt 6646-6904): sequence including the 3′ untranslated region of the nopaline synthase gene from the T-DNA of pTiT37 (Depicker et al., 1982).

gRNA Chimeric Gene
P-u6-3.1 (nt 6630-7145): The promoter region of the u6 gene of Oryza sativa (Jiang W. et al., 2013).
Guide RNA (nt 7146-7250): Sequence encoding a synthetic guide RNA for endonuclease CAS9-mediated DNA cleavage (Li et al., 2013), targeting epsps gene of Oryza sativa.
   SiteTS3 (nt 7146-7165): sgRNA targeting sequence
   sgR22 (nt 7146-7250): sequence encoding a synthetic guide RNA for endonuclease CAS9-mediated DNA cleavage (Li et al., 2013) targeting the Oryza sativa EPSPS gene
   sgRNA (nt 7166-7242): guide RNA scaffolding sequence
   TpolII (nt 7243-7250): RNA Polymerase III termination signal

Repair DNA for EPSPS Carrying TIPS Mutation
epspsOs-2Ga-4 (nt 404-1403): Fragment of genomic coding sequence of the modified 5-enolpyruvylshikimate-3-phosphate synthase gene of Oryza sativa, encoding for modified EPSPS protein of Oryza sativa species (unpublished)
   exon (nt 7570-7814):
   TIPS region (nt 7741-7779)
   T169I (nt 7762-7764)
   P173S (nt 7774-7776)
   Exon (nt 7894-8047)

Bar Selectable Marker Gene
P35S3 (nt8650-9435): sequence including the promoter region of the Cauliflower Mosaic Virus 35S transcript (Odell et al., 1985).
Bar coding sequence (nt9438-9989): coding sequence of the phosphinothricin acetyltransferase gene of Streptomyces hygroscopicus (Thompson et al., 1987).
3′nos (nt 10009-10269): sequence including the 3′ untranslated region of the nopaline synthase gene from the T-DNA of pTiT37 (Depicker et al., 1982).
LB (nt 10464-10488): left border repeat from the T-DNA of Agrobacterium tumefaciens (Zambryski, 1988).

### Example 2: Media

RSK-500=SK-1m salts (Khanna & Raina, 1998), Khanna vitamins (Khanna & Raina, 1998), L-proline 1.16 g/L, CuSO4.5H2O 2.5 mg/L, 2.4-D 2 mg/L, maltose 20 g/L, sorbitol 30 g/L, MES 0.5 g/L, agarose 6 g/L, pH 5.8

RSK-600=RSK500 medium but with 1 mg/L 2.4-D and 0.5 mg/L BAP

RSK-100=SK-1m salts (Khanna & Raina, 1998), Khanna vitamins (Khanna & Raina, 1998), L-proline 1.16 g/L, 2.4-D 2 mg/L, sucrose 30 g/L, MES 0.5 g/L, agarose 6 g/L, pH 5.8

RSK-201=SK-1m salts Duchefa (Khanna & Raina, 1998), Khanna vitamins (Khanna & Raina, 1998), L-proline 1.16 g/L, L-glutamine 0.8765 g/L, L-arginine 0.174 g/L, glycine 7.5 mg/L, L-aspartic acid 0.288 g/L, casein hydrolysate 300 mg/L, 2.4-D 2 mg/L, sucrose 20 g/L, mannitol 55 g/L, sorbitol 55 g/L MES 0.5 g/L, agarose 6 g/L, pH 5.8

MSR4=MS medium, L-proline 0.552 g/L, L-glutamine 0.8765 g/L, L-arginine 0.174 g/L, glycine 7.5 mg/L, L-aspartic acid 0.288 g/L, kinetin 1 mg/L, NM 0.5 mg/L, maltose 30 g/L, sorbitol 10 g/L, MES 0.5 g/L, agarose 6 g/L, pH 5.8

MSR2=MS medium, L-proline 0.552 g/L, casein hydrolysate 300 mg/L, NM 0.5 mg/L, sucrose 30 g/L, MES0.5 g/L, agarose 6 g/L, pH 5.8

AAM=AA medium (Hiei et al., 1994), L-glutamine 0.8765 g/L, L-arginine 0.174 g/L, glycine 7.5 mg/L, L-aspartic acid 0.288 g/L, casamino acids 500 mg/L, sucrose 68.5 g/L, glucose 36 g/L, pH 5.2

SKAS-1m preinduction medium=SK-1m salts Duchefa (Khanna & Raina, 1998), Khanna vitamins (Khanna & Raina, 1998), L-proline 1.16 g/L, L-glutamine 0.8765 g/L, L-arginine 0.174 g/L, glycine 7.5 mg/L, L-aspartic acid 0.288 g/L, casein hydrolysate 300 mg/L, 2.4-D 2 mg/L, acetosyringone 200 μM, sucrose 30 g/L, glucose 10 g/L, agarose 6 g/L, pH 5.2

SKAS-1m co-cultivation medium=SK-1m salts Duchefa (Khanna & Raina, 1998), Khanna vitamins (Khanna & Raina, 1998), 2.4-D 2 mg/L, acetosyringone 200 μM, sucrose 30 g/L, glucose 10 g/L, agarose 6 g/L pH 5.2

MS/2=MS medium with ½ concentration of MS salts, sucrose 30 g/L, agarose 4.5 g/L, pH 5.8

### REFERENCES

Khanna & Raina, Plant Cell, Tissue and Organ Culture, 52: 145-153, 1998
Hiei et al, Plant J., 6: 271-282, 1994

### Example 3: Particle Bombardment-Mediated Editing Using Embryogenic Callus

Mature seed derived embryogenic callus was used as starting material for particle bombardment. Hereto, sterilized seeds were incubated for ˜3 to 4 weeks on RSK100 substrate in the dark at a temperature between 25-30° C. for the induction of embryogenic callus.

After 3 to 4 weeks of incubation on RSK100, embryogenic callus is selected from the RSK100 plates and subcultured on RSK600 for a few days at a temperature between 25-30° C. under 16H light/8H dark photoperiod.

The day of bombardment, actively growing embryogenic pieces from the RSK600 plate are selected, cut in smaller pieces and transferred onto RSK201 for preplasmolysis for a few hours. After preplasmolysis the EC was bombarded using either the biolistic PDS-100/He particle delivery system (Bio-Rad), or the particle inflow gun (PIG) system (Grayel). With the Biorad system, the particle bombardment parameters were as follows: diameter gold particles, 0.3-3 μm; target distance, 9 cm; bombardment pressure, 9301.5 k Pa; gap distance, 6.4 mm; and macrocarrier flight distance, 11 mm. For each plasmid DNA (Cas9, gRNA, repair DNA) 0.125 pmol DNA was used per shot. With the Grayel system, the particle bombardment parameters were as follows: diameter gold particles, 0.6 μm; target distance 17 cm; bombardment pressure 500 k Pa; and for each plasmid DNA (Cas9, gRNA, repair DNA) 1.25 μg DNA was used per shot.

After bombardment, the callus pieces are transferred to non-selective RSK500 callus induction medium for a few days under a 16H light/8H dark photoperiod at a temperature between 25-30° C. After this period on non-selective substrate, the callus pieces are transferred to the RSK500 medium supplemented with 150 mg/L glyphosate as selective agent and incubated again under a 16H light/8H dark photoperiod at a temperature between 25-30° C.

After ˜3 to 4 weeks, the callus pieces showing proliferation of embryogenic callus on selective RSK500 medium with 150 mg/L glyphosate are subcultured on the same substrate. Each subcultivation should include extensive cutting of actively growing embryogenic callus pieces until a ‘pure’ glyphosate tolerant embryogenic callus line is obtained. The ‘pure’ active growing glyphosate tolerant embryogenic callus lines are then transferred to RSK600 substrate+150 mg/L glyphosate. After an incubation of about one month on RSK 600 medium, the callus pieces are transferred to non-selective regeneration medium MSR4 under a 16H light/8H dark photoperiod at 25-30° C. Shoot regenerating calli may be transferred to MSR2 medium for further development. Regenerating shoots are transferred to MS/2 substrate for further elongation prior to transfer to the greenhouse.

### Results

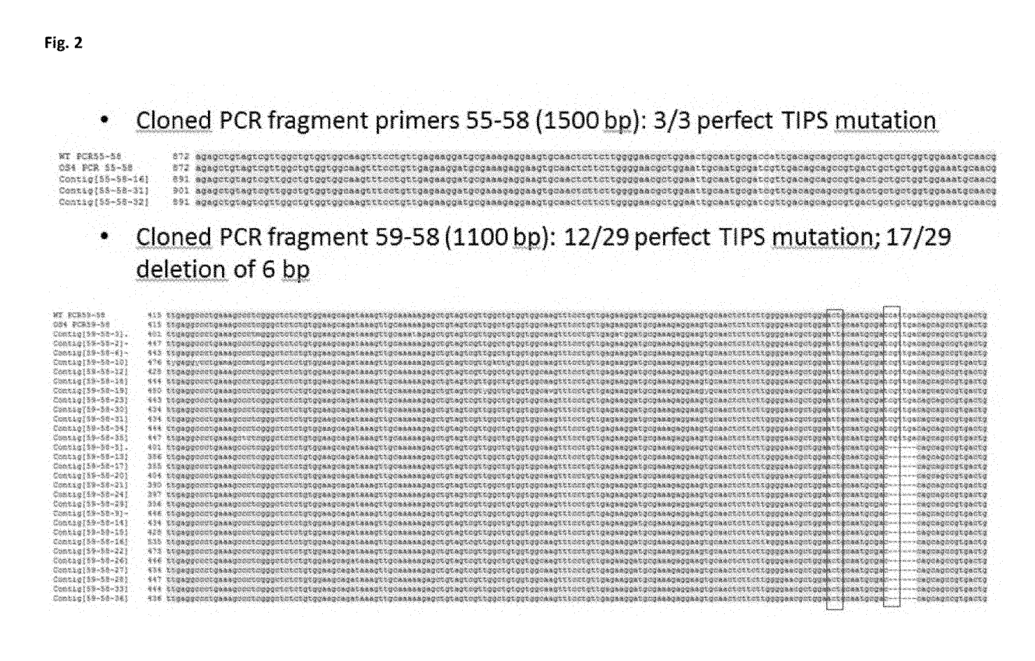

Embryogenic callus was transformed with plasmids pKVA790 (Cas9)+pKVA761 (repair DNA)+pKVA766 (gRNA) as described above. This resulted in the recovery of 1-8 GlyT events per ˜500 calli (0.2-1.6%) using the PIG device and 0-1 GlyT events per ˜500 calli (0-0.2%) using the Biorad device. Restriction digestion (Pvul) of the amplified PCR product over the target region was done as a first molecular screen to confirm the introduction of the TIPS mutation in the native epsps gene as a silent mutation to create a Pvul site was introduced close to the TIPS mutation in the donor DNA to facilitate molecular screening for identification of TIPS epsps edited events. Pvul digest of the amplified PCR product of 2 glyT callus events obtained by the Biorad gun reveal 2 mono-allelic TIPS edited events. Sequencing of cloned PCR products obtained from these 2 events showed that these were bi-allelic mutation events with the TIPS mutation in one allele and a 6 bp deletion at the target site in the other allele (See FIG. 2). Pvul digest of the amplified PCR product of 53 glyT callus events obtained by the PIG reveal 38 mono-allelic TIPS edited events, 14 bi-allelic TIPS edited events and 1 event with no TIPS mutation. Sequencing analysis on 13 bi-allelic events confirmed the presence of the TIPS mutation in both alleles. Sequencing of cloned PCR products obtained from 8 mono-allelic edited events obtained by the PIG showed that these were bi-allelic mutation events with the TIPS mutation in one allele and a non-specific mutation (insertion or deletion) in the other allele.

### Example 4: Agrobacterium-Mediated Editing Using Immature Embryos

### Methods

Freshly isolated immature embryos are transferred to solid SKAS-1m preinduction medium supplemented with acetylsalicylic acid 25 mg/L for 3-4 days in the dark at a temperature between 25-30° C. After preinduction the immature embryos are immersed in the Agrobacterium infection medium (2×10⁹ bact./ml in AAM+300 μM AS) for 10-15 minutes and then afterwards transferred to SKAS-1m cocultivation medium in the dark at ˜25° C. for 3 to 4 days.

After co-cultivation the coleoptile is removed from the embryos, and the embryos are transferred to non-selective RSK500 callus induction medium supplemented with 250 mg/L ticarcillin for a few days under a 16H light/8H dark photoperiod at a temperature between 25-30° C.

After this period on non-selective substrate, the embryos are transferred to the same RSK500 medium+250 mg/L ticarcillin and now supplemented with 150 mg/L glyphosate as selective agent and incubated again under a 16H light/8H dark photoperiod at a temperature between 25-30° C.

After 3 to 4 weeks, the embryos showing proliferation of embryogenic callus on selective RSK500 medium with 250 mg/L ticarcillin and 150 mg/L glyphosate, are subcultured on the same substrate. Each subcultivation should include extensive cutting of actively growing embryogenic callus pieces until a ‘pure’ glyphosate tolerant embryogenic callus line is obtained. The ‘pure’ active growing glyphosate tolerant embryogenic callus lines are then transferred to selective RSK600 medium with 250 mg/L ticarcillin and 150 mg/L glyphosate. After an incubation of about one month on RSK 600 medium, the callus pieces are transferred to non-selective regeneration medium MSR4 with 100 mg/L ticarcillin under a 16H light/8H dark photoperiod at a temperature between 25-30° C. Shoot regenerating calli may be transferred to MSR2 medium with 100 mg/L ticarcillin for further development. Regenerating shoots are transferred to MS/2 substrate for further elongation prior to transfer to the greenhouse.

### Results

The same components as described in Example 3, i.e. the Cas9 of pKVA790+the repair DNA of pKVA761+the gRNA of pKVA766 (gRNA), were cloned into one T-DNA vector pTKVA869/pBay00461 and transformed into Agrobacterium strain ACH5C3(GV400) which was subsequently used to transform immature embryos as described above.

This resulted in the recovery of ˜10% GlyTcallus events (77 glyT callus events/750 co-cultivated immature embryo's). Pvul digest of the amplified PCR product of 75 glyT callus events reveal 58 mono-allelic TIPS epsps edited events, 15 bi-allelic TIPS edited events and 1 event with no TIPS mutation. Sequencing analysis of the PCR product of 2 bi-allelic TIPS edited events confirmed the presence of the TIPS mutation in both alleles. Sequencing analysis of cloned PCR products from 6 mono-allelic TIPS edited events showed that these were bi-allelic mutation events with the TIPS mutation in one allele and a non-specific mutation (deletion or insertion) in the other allele.

### Example 5: Agrobacterium-Mediated Editing Using Embryogenic Callus

Mature seed derived embryogenic callus was used as starting material for the co-cultivation. Hereto, sterilized seeds were incubated for ˜3 to 4 weeks on RSK100 substrate in the dark at a temperature between 25-30° C. for the induction of embryogenic callus.

After 3 to 4 weeks of incubation on RSK100, embryogenic callus is selected from the RSK100 plates and subcultured on RSK600 for a few days at a temperature between 25-30° C. under 16H light/8H dark photoperiod.

The day of co-cultivation initiation, actively growing embryogenic pieces from the RSK600 plate are selected and immersed in the Agrobacterium infection medium (4×10⁹ bact./ml in MM+300 μM AS) for 10-15 minutes and then afterwards transferred to SKAS-1m cocultivation medium in the dark at ˜25° C. for 3 to 4 days.

After co-cultivation, the callus pieces are transferred to non-selective RSK500 callus induction medium+250 mg/L ticarcillin for a few days under a 16H light/8H dark photoperiod at a temperature between 25-30° C. After this period on non-selective substrate, the callus pieces are transferred to the same RSK500 medium+250 mg/L ticarcillin and now supplemented with 150 mg/L glyphosate as selective agent and incubated again under a 16H light/8H dark photoperiod at a temperature between 25-30° C.

After ˜3 to 4 weeks, the callus pieces showing proliferation of embryogenic callus on selective RSK500 medium+250 mg/L ticarcillin with 150 mg/L glyphosate are subcultured on the same substrate after intensive cutting. Each subcultivation should include extensive cutting of actively growing embryogenic callus pieces until a ‘pure’ glyphosate tolerant embryogenic callus line is obtained. The ‘pure’ active growing glyphosate tolerant embryogenic callus lines are then transferred to selective RSK600 substrate+250 mg/L ticarcillin and 150 mg/I glyphosate. After an incubation of about one month on RSK 600 medium, the callus pieces are transferred to regeneration medium MSR4 medium with 100 mg/L ticarcillin under a 16H light/8H dark photoperiod at a temperature between 25-30° C. Shoot regenerating may be transferred to MSR2 medium with 100 mg/L ticarcillin for further development. Regenerating shoots are transferred to MS/2 substrate for further elongation prior to transfer to the greenhouse.

### Results

The same components as described in Example 3, i.e. the Cas9 of pKVA790+the repair DNA of pKVA761+the gRNA of pKVA766 (gRNA), were cloned into one T-DNA vector pTKVA869/pBay00461 and transformed into Agrobacterium strain GA00182, which was subsequently used to transform embryogenic callus as described above.

This resulted in the recovery of 10-18 glyT events/˜500 co-cultivated callus pieces (2-4%). Pvul digest of the amplified PCR product of 10 glyT callus events revealed 9 mono-allelic TIPS epsps edited events and 1 bi-allelic TIPS edited event.

### Example 6: Agrobacterium-Mediated Editing Using Immature Embryos and Indirect Selection

### Methods

Freshly isolated immature embryos were transferred to solid SKAS-1m preinduction medium supplemented with acetylsalicylic acid 25 mg/L for 3-4 days in the dark at a temperature between 25-30° C. After preinduction the immature embryos were immersed in the Agrobacterium infection medium (2×10⁹ bact./ml in AAM+300 μM AS) for 10-15 minutes and then afterwards transferred to SKAS-1m cocultivation medium in the dark at ˜25° C. for 3 to 4 days.

After co-cultivation the coleoptile was removed from the embryos, and the embryos were transferred to non-selective RSK500 callus induction medium supplemented with 250 mg/L ticarcillin for a few days under a 16H light/8H dark photoperiod at a temperature between 25-30° C.

After 3 days on non-selective substrate, the embryos were transferred to the same RSK500 medium+250 mg/L ticarcillin and now supplemented with 5 mg/L phosphinothricin (PPT) as selective agent and incubated again under a 16H light/8H dark photoperiod at a temperature between 25-30° C.

After 3 to 4 weeks, the embryos showing proliferation of embryogenic callus on selective RSK500 medium with 250 mg/L ticarcillin and 5 mg/L PPT, were cut in smaller pieces and subcultured on the same substrate with 5 mg/L PPT. After 3 weeks proliferating callus sectors on PPT were subcultured once again on selective RSK500 medium with 250 mg/L ticarcillin and 5 mg/L PPT.

### Results

Agrobacterium strain ACH5C3(GV400) as described in Example 4, comprising the three component T-DNA vector pBay00461/pTKVA869 in addition to the bar selectable marker as described in Example 1, was used to transform immature embryos and subjected to PPT selection as described above.

From the initial 178 plated immature embryo's (IEs), 95 IEs produced PPT tolerant calli. From these actively growing PPT tolerant embryogenic callus lines, multiple sectors were harvested for conducting a TIPS PCR with one primer over the TIPS mutation and one primer in the epsps gene outside of the region of homology of the donor DNA. In 44 out of the 95 responding IEs (46.3%), one or more callus sectors yielded a TIPS-specific PCR amplification product.

From a subset of the responding IEs, the remaining EC after sampling for TIPS PCR has been chopped in small pieces and glyphosate tolerant events could still be recovered by selection on 150 mg/L glyphosate.

This indicates that co-delivery of a selectable marker gene using one Agrobacterium strain and subsequent selection on the corresponding selective agent allows the recovery of repair DNA-mediated edited events without “direct” selection on the edit itself (as described in Example 4 and 5).

When co-cultivating with two Agro-strains, one containing the repair DNA plus the gRNA- and Cas9-expression cassettes, the other comprising the selectable marker gene (bar), after PPT selection as described above, of the initial plated 135 immature embryos, three gly^{R} events were recovered.

1. A method for modifying the (nuclear) genome of a plant cell at a preselected site or for producing a plant cell with a modified genome comprising the steps of:
a. introducing into said plant cell an RNA-guided endonuclease (RGEN) and at least one guide polynucleotide, wherein said RGEN and said at least one guide polynucleotide are capable of forming a complex that enables the RGEN to introduce a (double stranded) DNA break or one or more nicks or single stranded breaks, or to induce DNA strand displacement, at or near said preselected site;
b. introducing into said cell at least one donor polynucleotide comprising a polynucleotide of interest;
c. selecting a plant cell wherein said donor polynucleotide has been used as a template for repair of said DNA break, thereby integrating said polynucleotide of interest at said preselected site and resulting in a modification of said genome at said preselected site, wherein said modification is selected from
i. a replacement of at least one nucleotide;
ii. a deletion of at least one nucleotide;
iii. an insertion of at least one nucleotide; or
iv. any combination of i.-iii.
characterised in that said RGEN, said at least one guide polynucleotide and said at least one donor polynucleotide are introduced into said plant cell by contacting said plant cell with at least one bacterium comprising a chimeric gene encoding said RGEN, at least one chimeric gene encoding said at least one guide polynucleotide and said at least one donor polynucleotide.

2. The method of claim 1, wherein said bacterium is Agrobacterium tumefaciens.

3. The method of claim 1 or 2, wherein said chimeric gene encoding said endonuclease, said at least one chimeric gene encoding said at least one guide polynucleotide and said at least one donor polynucleotide are located on one T-DNA vector.

4. The method of any one of claims 1-3, wherein said chimeric gene encoding said endonuclease, said at least one chimeric gene encoding said at least one guide polynucleotide and said at least one donor polynucleotide are located on one T-DNA molecule (between a single set of T-DNA borders).

5. The method of any one of claims 1-4, wherein said RGEN is a nickase or a pair of nickases.

6. The method of any one of claims 1-4, wherein said RGEN is Cas9 or Cpf1.

7. The method of any one of claims 1-6, wherein said chimeric gene encoding said at least one guide polynucleotide encodes two or more guide polynucleotide sequences.

8. The method of any one of claims 1-7, wherein the coding region of said chimeric gene encoding said endonuclease has been optimized for expression in a plant.

9. The method of any one of claims 1 to 8, wherein said RGEN comprises the amino acid sequence of SEQ ID NO 6 from amino acid at position 10 to amino acid at position 1388.

10. The method of any one of claims 1 to 8, wherein said chimeric gene encoding said RGEN comprises the nucleotide sequence of SEQ ID NO. 5 from nucleotide 28 to nucleotide 4164.

11. The method of any one of claims 1-10, wherein said bacterium further comprises a selectable marker gene that is introduced into and expressed in said plant cell.

12. The method of claim 11, wherein said selectable marker gene confers upon said plant cell a selectable phenotype.

13. The method of claim 11 or 12, wherein said selectable marker gene is located on said one T-DNA vector.

14. The method of any one of claims 11-13, wherein said selectable marker gene is located on said one T-DNA molecule.

15. The method of any one of claims 1-14, wherein said modification in said nuclear genome confers upon said plant cell a selectable phenotype.

16. The method of any one of claims 12-15, wherein said selectable phenotype is tolerance to one or more herbicides.

17. The method of any one of claims 12-16, wherein said selectable phenotype conferred to said plant cell by said modification can be used for direct selection of a plant cell comprising said modification.

18. The method of any one of claims 12-17, wherein the selectable phenotype conferred to said plant cell by said selectable marker gene can be used to select a plant cell comprising said modification.

19. The method of any one of claims 1-18 wherein said cell is comprised within an immature embryo or embryogenic callus.

20. The method of any one of claims 1 to 19, wherein said donor DNA molecule comprises one or two flanking nucleotide sequences flanking the DNA molecule of interest, said flanking nucleotide sequence or sequences having sufficient homology to the genomic DNA upstream and/or downstream of said preselected site to allow recombination with said upstream and/or downstream DNA region.

21. The method of any one of claims 1-20, wherein said polynucleotide of interest comprises one or more expressible gene(s) of interest, said expressible gene of interest optionally being selected from the group of a herbicide tolerance gene, an insect resistance gene, a disease resistance gene, an abiotic stress resistance gene, an enzyme involved in oil biosynthesis, carbohydrate biosynthesis, an enzyme involved in fiber strength or fiber length, an enzyme involved in biosynthesis of secondary metabolites.

22. The method of any one of claims 1-21, comprising the further step of growing said selected plant cell comprising said modification into a plant.

23. The method of claim 22, comprising the further step of crossing said plant with another plant and optionally obtaining a progeny plant comprising said modification.

24. The method of claim 22 or 23, comprising the further step of selecting a progeny plant comprising said modification but not comprising said chimeric gene encoding said RGEN, said at least one chimeric gene encoding said at least one guide polynucleotide and said selectable marker gene.

25. The method of any one of claims 1-24, wherein said plant cell or plant is a rice plant cell or plant.

26. A plant cell, plant part, seed, plant product or plant comprising a modification at a preselected site in the (nuclear) genome produced by the method of any one of claims 1-25.

27. A bacterium comprising a chimeric gene encoding an RGEN, at least one chimeric gene encoding at least one guide polynucleotide and at least one donor polynucleotide as described in any one of claims 1-21, wherein said bacterium is capable of transferring or introducing said chimeric gene encoding said RGEN, said chimeric gene encoding said guide polynucleotide and said donor polynucleotide into (the nuclear genome of) a plant cell, wherein said RGEN and said guide polynucleotide upon expression in said plant cell are capable of forming a complex that enables the RGEN to introduce a DNA break at a preselected site in the (nuclear) genome of a plant cell and wherein said donor polynucleotide is to be used as a template for repair of said DNA break.

28. The bacterium of claim 27, which is Agrobacterium tumefaciens.

29. The bacterium of claim 27 or 28, wherein said chimeric gene encoding said RGEN, said chimeric gene encoding said guide polynucleotide and said donor polynucleotide are located on one vector, optionally on one T-DNA molecule (between a pair of T-DNA borders).

30. A (T-DNA) vector comprising the chimeric gene encoding an RGEN, the at least one chimeric gene encoding at least one guide polynucleotide and the at least one donor polynucleotide as described in any one of claims 1-21, optionally on one T-DNA molecule (between a pair of T-DNA borders).

31. The bacterium of any one of claims 27-29, or the vector of claim 30, further comprising a selectable marker gene, optionally on said on one T-DNA molecule (between a pair of T-DNA borders)

32. A method for modifying an endogenous EPSPS gene in a plant cell, or for producing a plant cell having a modified EPSPS gene, or for testing the efficiency of genome editing (components), comprising the steps of:
a. expressing in said cell a site-directed DNA modifying polypeptide recognising a sequence in an endogenous EPSPS gene of said plant and/or introducing into said plant cell a donor polynucleotide that can be used as a template for modifying said endogenous EPSPS gene;
b. evaluating tolerance of said plant cell to one or more EPSPS inhibitors by culturing said plant cell on medium comprising said EPSPS inhibitor(s); and optionally
c. selecting a plant cell having increased tolerance to said EPSPS inhibitor.

33. The method of claim 32, wherein said EPSPS inhibitor is used as a first selective agent.

34. The method of claim 32 or 33, wherein said plant cell is a rice plant cell.

35. A method for modifying the (nuclear) genome of a plant cell at a preselected site comprising the steps of:
a. introducing into said cell a nucleotide-guided DNA modifying polypeptide (NGDMP) and a guide polynucleotide, wherein said NGDMP and guide polynucleotide are capable of forming a complex that enables the NGDMP to modify the genome of a plant cell at a preselected site;
b. selecting a plant cell wherein said genome has been modified at said preselected site characterised in that said NGDMP, said guide polynucleotide are introduced to said plant cell using a particle inflow gun.

36. A method for modifying the (nuclear) genome of a plant cell at a preselected site comprising the steps of:
a. introducing into said cell a nucleotide-guided DNA modifying polypeptide (NGDMP) and a guide polynucleotide, wherein said NGDMP and guide polynucleotide are capable of forming a complex that enables the NGDMP to modify the genome of a plant cell at a preselected site;
b. introducing into said cell at least one (plant-expressible) selectable marker gene;
c. selecting one or more plant cells comprising said selectable marker gene)
d. selecting a plant cell wherein said genome has been modified at said preselected site characterised in that said NGDMP, said at least one guide polynucleotide and said at least one selectable marker gene are introduced into said plant cell by contacting said plant cell with at least one bacterium comprising a chimeric gene encoding said RGEN, at least one chimeric gene encoding said at least one guide polynucleotide and at least one polynucleotide comprising said selectable marker gene.

37. The method of claim 35 or 36, wherein said RGDMP is an RGEN, said RGEN and said at least one guide polynucleotide being capable of forming a complex that enables the RGEN to introduce a DNA break at or near said preselected site.

38. The method of claim 37, wherein together with said RGEN and said guide polynucleotide a donor polynucleotide comprising a polynucleotide of interest is introduced into said plant cell, wherein said donor polynucleotide is used as a template for repair of said DNA break, thereby integrating said polynucleotide of interest at said preselected site and resulting in a modification of said genome at said preselected site.

39. A bacterium comprising a chimeric gene encoding an NGDMP, at least one chimeric gene encoding at least one guide polynucleotide and at least one (plant-expressible) selectable marker gene as described in any one of claims 36-38, wherein said bacterium is capable of transferring or introducing said chimeric gene encoding said NGDMP, said chimeric gene encoding said guide polynucleotide and said selectable marker gene into (the nuclear genome of) a plant cell, wherein said NGDMP and said guide polynucleotide upon expression in said plant cell are capable of forming a complex that enables the NGDMP to modify the (nuclear) genome of a plant cell.

40. The bacterium of claim 39, which is Agrobacterium tumefaciens.

41. The bacterium of claim 39 or 40, wherein said chimeric gene encoding said NGDMP, said chimeric gene encoding said guide polynucleotide and said selectable marker gene are located on one vector, optionally on one T-DNA molecule (between a pair of T-DNA borders).

42. A (T-DNA) vector comprising the chimeric gene encoding an NGDMP, the chimeric gene encoding a guide polynucleotide and the selectable marker gene as described in any one of claims 36-41, optionally on one T-DNA molecule (between a pair of T-DNA borders).


SEQUENCE LISTING (6 sequences)

SEQ ID NO: 1 - DNA, 9186 bp, Artificial Sequence
  Cas 9-vector pBAY00201
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgggcccggccggccgcgatcgcgcggccgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatgcttagggcccggtagttctacttctgtccatgtttgtgttagatccgtgtttgtgttagatccgtgctactagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagccatggcccctaagaagaaacgaaaggtaggcatccacggtgttccagcggctatggacaagaagtacagcatcgggctggacattggcacgaactctgttgggtgggcggttatcactgacgagtacaaggtgccgtcgaagaagttcaaggtgctaggcaacaccgaccgtcacagcatcaagaagaacctcatcggcgccctgttgtttgactctggggaaaccgccgaggctactcgactaaagagaaccgcccgtaggcgatacactaggcggaagaacaggatctgttatctccaggagatcttcagcaacgagatggcgaaggtcgatgactcattcttccaccgccttgaggagtctttcctggtcgaggaggacaaaaagcacgagcgacacccgatcttcgggaacatcgttgatgaggtagcctaccacgagaagtaccctactatctaccacctgcgcaagaaactagtggactccaccgacaaggcggacctccgccttatctatctcgccctcgcccacatgatcaagtttcgcgggcacttcctcatcgaaggcgatctcaatccggacaacagcgacgtggacaagctattcatccagctagttcagacctacaaccagctctttgaagagaaccctattaacgcgagcggggtggacgccaaagccatcctttccgccaggctctctaagagccggagactcgagaaccttatcgctcagttgcccggcgagaagaaaaacggccttttcggcaacctgattgccctgtccttgggcttgacgccgaacttcaagagcaacttcgacctagctgaggatgctaaactgcagctgtccaaggacacttatgatgacgaccttgacaacctcctggcccaaattggggatcagtacgcggacctcttcctcgctgccaagaacctctccgacgcgattctgttgtcggacatcctccgcgtgaacaccgagattactaaggccccactgtccgcgagcatgatcaaacgctacgacgagcaccatcaggatctaactctgctcaaggcgctcgtccgtcagcaactccctgagaagtacaaggagatcttcttcgaccagtccaagaacggctacgccggttacattgatggtggcgcgagccaggaggagttctacaagttcatcaagcccatcttagagaagatggacggcaccgaagagcttctcgtgaagctcaacagggaggacctcctaaggaagcagcgcacctttgacaacgggtctattcctcatcagatccatctcggcgagctccacgcgattttgagacgccaagaggacttctacccgttcttgaaggacaaccgggagaagatcgagaagatcctcacgttcaggatcccttattacgtcggccccctagctaggggtaactcgagattcgcgtggatgaccagaaagtccgaggaaaccattaccccgtggaactttgaagaggtggtcgacaagggcgcgagcgcgcaatcgttcattgagcggatgaccaacttcgacaagaacctaccgaacgagaaggtcctgccgaaacactccctgctctacgagtacttcaccgtttataacgagctgacgaaggtcaagtacgtgactgagggcatgcgtaaaccagcgttcctcagcggtgagcagaagaaggctatcgtggacctgctctttaagacgaaccgcaaggtgaccgtgaagcagctcaaggaggattatttcaagaagatcgagtgcttcgactcagtggagattagcggcgtggaggaccgtttcaatgcgagcttaggtacctaccacgacctcctgaagatcatcaaggacaaggacttcctcgacaacgaggaaaacgaggacatcctggaggacatcgtgctcaccctgaccctctttgaagacagggagatgatcgaggaaaggttgaagacctacgcgcacctattcgacgacaaggtcatgaaacagctcaagcggaggagatacactggctggggcagactttcgcggaagctcatcaatgggatcagggacaagcagtccggcaagaccatcctggacttcttgaagagcgacggcttcgctaatcgcaacttcatgcagctcattcatgacgactccctaacattcaaggaggacatccaaaaggcgcaagtctcgggtcagggggattctttgcacgagcacatcgcaaacctcgccggttctccggccattaaaaagggcatccttcagacggtcaaggtggtcgacgagctcgtgaaggttatgggccggcataagcccgagaacatagtgatcgagatggctagggagaaccagaccacgcaaaagggccagaagaactcacgtgagcgcatgaagaggatcgaggaaggtatcaaggagctgggctctcagattctcaaggagcatccggtcgagaacacccagcttcagaacgagaagctgtacctctactacctccagaatggccgcgacatgtacgttgatcaggagttagatattaatcggctgagcgactacgacgttgaccacatcgtgccccagtcgttcctcaaggatgactccatcgacaacaaggtcctgaccagatcggacaagaaccgtggtaagagcgacaatgtgccctccgaggaggtggtcaaaaagatgaagaactattggcggcagttgctgaacgccaagctcatcacccagcgaaagttcgataatttaacgaaggccgaacgagggggcctttccgaactcgataaggccggtttcatcaagcgtcagctggttgagacgcgacagatcacgaagcatgtggcccagattctggacagccggatgaacacgaagtacgacgagaacgacaaacttattcgggaggtgaaggtgataaccctcaagtccaagttagtgtcggacttccgcaaggactttcagttctataaagtgcgcgagatcaacaattaccaccacgcccacgacgcctacttgaatgccgtggtcggcacggcgctcatcaaaaagtaccccaagctggagagcgagttcgtctacggcgactacaaggtttatgacgtcaggaagatgattgcgaagtcagagcaagagatcgggaaggccaccgccaagtacttcttctactcgaacatcatgaacttctttaagaccgagatcaccctcgccaatggggagatccggaaacgtccccttatcgagacgaacggcgagactggcgagatagtctgggacaaaggtcgcgatttcgccaccgttcgaaaggtgctctcaatgcctcaggtcaatatcgtgaagaagaccgaggttcagacggggggcttcagcaaggagtccatcctccctaagcgcaactctgacaagctgatcgcccggaaaaaggattgggacccgaaaaagtacggcggcttcgattctcccaccgtagcttatagcgtgctcgtggttgccaaggtggagaagggcaagagcaagaaactgaagtcggtcaaggagttactcggcatcacaatcatggagcgcagctcgtttgaaaagaacccgatcgacttcctggaggccaagggctataaggaggtgaagaaggacctcatcattaagctgccgaagtacagcttgttcgagctcgagaatggcaggaagaggatgctagcttcggccggcgaacttcaaaaagggaacgagctcgcgttgccgtcgaagtacgtgaacttcctctacttagctagtcactatgaaaagctgaagggctcgccggaggacaacgagcagaagcagctcttcgtcgagcaacacaagcactacctcgacgagatcatcgagcagataagtgagttcagcaagcgcgtgatcctcgctgatgccaacctcgataaggtgctcagcgcctacaataagcaccgagacaagccgattcgggagcaggcggagaacatcattcatttatttacgctcactaacctaggcgctccggctgccttcaagtacttcgacaccacgatcgacaggaagcgctacaccagcaccaaagaagtcctcgacgcgacgctcattcaccagagcatcacagggctctacgagactcggatcgacttgtcgcaattgggtggggataagaggccggccgcgactaaaaaggccggccaagccaagaaaaagaaatgattgctagcacgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctgcaggcccgggttaattaaatttaaatggcgcgccagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 2 - DNA, 3318 bp, Artificial Sequence
  gRNA vector pKVA766
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggaaccctgcaggaaaaaaaaagcaccgactcggtgccactttttcaagttgataacggactagccttattttaacttgctatttctagctctaaaacttgacagcagccgtgactgcaagctgactacagcgcgcgggtttataagctggttccatcgtctttcggtttccgatgtggtactaaacatcccccgcaacgccaccgcgcaaaccatcggatccccatccaacggactaaagtgggtagcagcgccacccgcgcggtacggcgcgcgtgggctgcaccttccgacgcagcccaaatcaggatacggcccattagtatatattctcctcctcccagcccacatcacatgcgtttcacactttcgcctgtaatcgctcaagtagtcaagtatgagctctgtctgcacgaactgattgtcactagtgtaccatcaaactactagacgtgacttgtgataatctcagacgcttatggagtgaaacatggtaaaaaaatgcgtttatgtgcttgatcactttgatcagccttgcctccatcgtctgaaacatgataagaagacctttgttacgaaagtgtgaaacgattctgttattccttcggctatgccgtaattcaactttcacaaagcgatcgcgttccatcccaatggcgcgccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 3 - DNA, 3645 bp, Artificial Sequence
  TIPS repair vector pKVA761
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcgatcgcttctcccaaaattatgcaattttgaggctcctctacatcattataattccccaatacattgctctttattcttaacagctttgatcgcgaaatttaacattttaattcttgagctgttattttgtagcatcagtttatcatgagccatgtttggtactaaatatacaatcccttgggtttatttgtttccaagcatgtcattaacttatcttaatgtggacaagaaactgatgcctgcttacattgctattatttcaagcgggtattgatcctttgacatgtgattgatcatttttttttctctggttattagggcacaacagtggtggacaacttgctgaacagtgaggatgttcactacatgcttgaggccctgaaagccctcgggctctctgtggaagcagataaagttgcaaaaagagctgtagtcgttggctgtggtggcaagtttcctgttgagaaggatgcgaaagaggaagtgcaactcttcttggggaacgctggaattgcaatgcgatcgttgacagcagccgtgactgctgctggtggaaatgcaacgtatgtttttttttaatgtttatgaaaataagtatggaattcatggggtatgttttatgacctttttctttaccatcagttatgtgcttgatggagtgccacgaatgagggagagaccgattggtgacttggttgtcgggttgaaacaacttggtgcggatgtcgactgtttccttggcactgaatgcccacctgttcgtgtcaagggaattggaggacttcctggtggcaaggttagttactcctaaactgcatcctttgtacttctgtatgcacctcaattctttgtcaaccttctgcatttataaggaacattctatgatgaaattcgaccttacactgcacagtaacttgaaatgtttcatgcttaatcaatatgccatattcctgccaagctcaagcgagcaatatttgtttgaatttggtaccatatttttgtatacctgcaggggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 4 - DNA, 16730 bp, Artificial Sequence
  T-DNA vector pBAY00461
aattacaacggtatatatcctgccagtactcggccgtcgaccgcggtaccccggaattttgtggcgctctatcatagctataaacctattcagcacaatattagttagctaggggccccctcgagggcgatcgcgcggccgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatgcttagggcccggtagttctacttctgtccatgtttgtgttagatccgtgtttgtgttagatccgtgctactagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcagccatggcccctaagaagaaacgaaaggtaggcatccacggtgttccagcggctatggacaagaagtacagcatcgggctggacattggcacgaactctgttgggtgggcggttatcactgacgagtacaaggtgccgtcgaagaagttcaaggtgctaggcaacaccgaccgtcacagcatcaagaagaacctcatcggcgccctgttgtttgactctggggaaaccgccgaggctactcgactaaagagaaccgcccgtaggcgatacactaggcggaagaacaggatctgttatctccaggagatcttcagcaacgagatggcgaaggtcgatgactcattcttccaccgccttgaggagtctttcctggtcgaggaggacaaaaagcacgagcgacacccgatcttcgggaacatcgttgatgaggtagcctaccacgagaagtaccctactatctaccacctgcgcaagaaactagtggactccaccgacaaggcggacctccgccttatctatctcgccctcgcccacatgatcaagtttcgcgggcacttcctcatcgaaggcgatctcaatccggacaacagcgacgtggacaagctattcatccagctagttcagacctacaaccagctctttgaagagaaccctattaacgcgagcggggtggacgccaaagccatcctttccgccaggctctctaagagccggagactcgagaaccttatcgctcagttgcccggcgagaagaaaaacggccttttcggcaacctgattgccctgtccttgggcttgacgccgaacttcaagagcaacttcgacctagctgaggatgctaaactgcagctgtccaaggacacttatgatgacgaccttgacaacctcctggcccaaattggggatcagtacgcggacctcttcctcgctgccaagaacctctccgacgcgattctgttgtcggacatcctccgcgtgaacaccgagattactaaggccccactgtccgcgagcatgatcaaacgctacgacgagcaccatcaggatctaactctgctcaaggcgctcgtccgtcagcaactccctgagaagtacaaggagatcttcttcgaccagtccaagaacggctacgccggttacattgatggtggcgcgagccaggaggagttctacaagttcatcaagcccatcttagagaagatggacggcaccgaagagcttctcgtgaagctcaacagggaggacctcctaaggaagcagcgcacctttgacaacgggtctattcctcatcagatccatctcggcgagctccacgcgattttgagacgccaagaggacttctacccgttcttgaaggacaaccgggagaagatcgagaagatcctcacgttcaggatcccttattacgtcggccccctagctaggggtaactcgagattcgcgtggatgaccagaaagtccgaggaaaccattaccccgtggaactttgaagaggtggtcgacaagggcgcgagcgcgcaatcgttcattgagcggatgaccaacttcgacaagaacctaccgaacgagaaggtcctgccgaaacactccctgctctacgagtacttcaccgtttataacgagctgacgaaggtcaagtacgtgactgagggcatgcgtaaaccagcgttcctcagcggtgagcagaagaaggctatcgtggacctgctctttaagacgaaccgcaaggtgaccgtgaagcagctcaaggaggattatttcaagaagatcgagtgcttcgactcagtggagattagcggcgtggaggaccgtttcaatgcgagcttaggtacctaccacgacctcctgaagatcatcaaggacaaggacttcctcgacaacgaggaaaacgaggacatcctggaggacatcgtgctcaccctgaccctctttgaagacagggagatgatcgaggaaaggttgaagacctacgcgcacctattcgacgacaaggtcatgaaacagctcaagcggaggagatacactggctggggcagactttcgcggaagctcatcaatgggatcagggacaagcagtccggcaagaccatcctggacttcttgaagagcgacggcttcgctaatcgcaacttcatgcagctcattcatgacgactccctaacattcaaggaggacatccaaaaggcgcaagtctcgggtcagggggattctttgcacgagcacatcgcaaacctcgccggttctccggccattaaaaagggcatccttcagacggtcaaggtggtcgacgagctcgtgaaggttatgggccggcataagcccgagaacatagtgatcgagatggctagggagaaccagaccacgcaaaagggccagaagaactcacgtgagcgcatgaagaggatcgaggaaggtatcaaggagctgggctctcagattctcaaggagcatccggtcgagaacacccagcttcagaacgagaagctgtacctctactacctccagaatggccgcgacatgtacgttgatcaggagttagatattaatcggctgagcgactacgacgttgaccacatcgtgccccagtcgttcctcaaggatgactccatcgacaacaaggtcctgaccagatcggacaagaaccgtggtaagagcgacaatgtgccctccgaggaggtggtcaaaaagatgaagaactattggcggcagttgctgaacgccaagctcatcacccagcgaaagttcgataatttaacgaaggccgaacgagggggcctttccgaactcgataaggccggtttcatcaagcgtcagctggttgagacgcgacagatcacgaagcatgtggcccagattctggacagccggatgaacacgaagtacgacgagaacgacaaacttattcgggaggtgaaggtgataaccctcaagtccaagttagtgtcggacttccgcaaggactttcagttctataaagtgcgcgagatcaacaattaccaccacgcccacgacgcctacttgaatgccgtggtcggcacggcgctcatcaaaaagtaccccaagctggagagcgagttcgtctacggcgactacaaggtttatgacgtcaggaagatgattgcgaagtcagagcaagagatcgggaaggccaccgccaagtacttcttctactcgaacatcatgaacttctttaagaccgagatcaccctcgccaatggggagatccggaaacgtccccttatcgagacgaacggcgagactggcgagatagtctgggacaaaggtcgcgatttcgccaccgttcgaaaggtgctctcaatgcctcaggtcaatatcgtgaagaagaccgaggttcagacggggggcttcagcaaggagtccatcctccctaagcgcaactctgacaagctgatcgcccggaaaaaggattgggacccgaaaaagtacggcggcttcgattctcccaccgtagcttatagcgtgctcgtggttgccaaggtggagaagggcaagagcaagaaactgaagtcggtcaaggagttactcggcatcacaatcatggagcgcagctcgtttgaaaagaacccgatcgacttcctggaggccaagggctataaggaggtgaagaaggacctcatcattaagctgccgaagtacagcttgttcgagctcgagaatggcaggaagaggatgctagcttcggccggcgaacttcaaaaagggaacgagctcgcgttgccgtcgaagtacgtgaacttcctctacttagctagtcactatgaaaagctgaagggctcgccggaggacaacgagcagaagcagctcttcgtcgagcaacacaagcactacctcgacgagatcatcgagcagataagtgagttcagcaagcgcgtgatcctcgctgatgccaacctcgataaggtgctcagcgcctacaataagcaccgagacaagccgattcgggagcaggcggagaacatcattcatttatttacgctcactaacctaggcgctccggctgccttcaagtacttcgacaccacgatcgacaggaagcgctacaccagcaccaaagaagtcctcgacgcgacgctcattcaccagagcatcacagggctctacgagactcggatcgacttgtcgcaattgggtggggataagaggccggccgcgactaaaaaggccggccaagccaagaaaaagaaatgattgctagcacgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccctgcaggtttgtgaaagttgaattacggcatagccgaaggaataacagaatcgtttcacactttcgtaacaaaggtcttcttatcatgtttcagacgatggaggcaaggctgatcaaagtgatcaagcacataaacgcatttttttaccatgtttcactccataagcgtctgagattatcacaagtcacgtctagtagtttgatggtacactagtgacaatcagttcgtgcagacagagctcatacttgactacttgagcgattacaggcgaaagtgtgaaacgcatgtgatgtgggctgggaggaggagaatatatactaatgggccgtatcctgatttgggctgcgtcggaaggtgcagcccacgcgcgccgtaccgcgcgggtggcgctgctacccactttagtccgttggatggggatccgatggtttgcgcggtggcgttgcgggggatgtttagtaccacatcggaaaccgaaagacgatggaaccagcttataaacccgcgcgctgtagtcagcttgcagtcacggctgctgtcaagttttagagctagaaatagcaagttaaaataaggctagtccgttatcaacttgaaaaagtggcaccgagtcggtgctttttttttaattaattctcccaaaattatgcaattttgaggctcctctacatcattataattccccaatacattgctctttattcttaacagctttgatcgcgaaatttaacattttaattcttgagctgttattttgtagcatcagtttatcatgagccatgtttggtactaaatatacaatcccttgggtttatttgtttccaagcatgtcattaacttatcttaatgtggacaagaaactgatgcctgcttacattgctattatttcaagcgggtattgatcctttgacatgtgattgatcatttttttttctctggttattagggcacaacagtggtggacaacttgctgaacagtgaggatgttcactacatgcttgaggccctgaaagccctcgggctctctgtggaagcagataaagttgcaaaaagagctgtagtcgttggctgtggtggcaagtttcctgttgagaaggatgcgaaagaggaagtgcaactcttcttggggaacgctggaattgcaatgcgatcgttgacagcagccgtgactgctgctggtggaaatgcaacgtatgtttttttttaatgtttatgaaaataagtatggaattcatggggtatgttttatgacctttttctttaccatcagttatgtgcttgatggagtgccacgaatgagggagagaccgattggtgacttggttgtcgggttgaaacaacttggtgcggatgtcgactgtttccttggcactgaatgcccacctgttcgtgtcaagggaattggaggacttcctggtggcaaggttagttactcctaaactgcatcctttgtacttctgtatgcacctcaattctttgtcaaccttctgcatttataaggaacattctatgatgaaattcgaccttacactgcacagtaacttgaaatgtttcatgcttaatcaatatgccatattcctgccaagctcaagcgagcaatatttgtttgaatttggtaccatatttttgtataggcgcgccgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggaattcgatatcattaccctgttatccctaaagcttattaatataacttcgtatagcatacattatacgaagttatgtttcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctgaaatcaccagtctctctctataaatctatctctctctctataacaatggacccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatctgagatcacccgttctaggatccgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgaaacataacttcgtatagcatacattatacgaagttatcaaaacgtcgtgagacagtttggttaactataacggtcctaaggtagcgattagttagctagcgagcggcgaactaataactccgctctaccgaaagttactcgaggcattacggcattacggcactcgcgagggtcccaattcgagcatggagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggacttttaaacatcatccgtcggatggcgttgcgagagaagcagtcgatccgtgagatcagccgacgcaccgggcaggcgcgcaacacgatcgcaaagtatttgaacgcaggtacaatcgagccgacgttcacggtaccggaacgaccaagcaagctagcttagtaaagccctcgctagattttaatgcggatgttgcgattacttcgccaactattgcgataacaagaaaaagccagcctttcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggacagttgcgtgagcgcatacgctacttgcattacagcttacgaaccgaacaggcttatgtccactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtctctagagctctagagggttcgcatcctcggttttctggaaggcgagcatcgtttgttcgcccagcttctgtatggaacgggcatgcggatcagtgagggtttgcaactgcgggtcaaggatctggatttcgatcacggcacgatcatcgtgcgggagggcaagggctccaaggatcgggccttgatgttacccgagagcttggcacccagcctgcgcgagcaggatcgatccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgatagctcttgatccggcaaacaaaccaccgctggtagcggtggtttttttgtttgcaagcagcagattacgcgcagaaaaaaaggatctcaagaagatccggaaaacgcaagcgcaaagagaaagcaggtagcttgcagtgggcttacatggcgatagctagactgggcggttttatggacagcaagcgaaccggaattgccagattcgaagctcggtcccgtgggtgttctgtcgtctcgttgtacaacgaaatccattcccattccgcgctcaagatggcttcccctcggcagttcatcagggctaaatcaatctagccgacttgtccggtgaaatgggctgcactccaacagaaacaatcaaacaaacatacacagcgacttattcacacgcgaca

SEQ ID NO: 5 - DNA, 4206 bp, Artificial Sequence
  Modified Cas 9 coding region comprising nuclear localization signals and codon optimized
  misc_feature (10)..(27): nuclear localization signal Large T-antigen SV40
  misc_feature (4165)..(4203): nuclear localization signal nucleoplasmin
atggcccctaagaagaaacgaaaggtaggcatccacggtgttccagcggctatggacaagaagtacagcatcgggctggacattggcacgaactctgttgggtgggcggttatcactgacgagtacaaggtgccgtcgaagaagttcaaggtgctaggcaacaccgaccgtcacagcatcaagaagaacctcatcggcgccctgttgtttgactctggggaaaccgccgaggctactcgactaaagagaaccgcccgtaggcgatacactaggcggaagaacaggatctgttatctccaggagatcttcagcaacgagatggcgaaggtcgatgactcattcttccaccgccttgaggagtctttcctggtcgaggaggacaaaaagcacgagcgacacccgatcttcgggaacatcgttgatgaggtagcctaccacgagaagtaccctactatctaccacctgcgcaagaaactagtggactccaccgacaaggcggacctccgccttatctatctcgccctcgcccacatgatcaagtttcgcgggcacttcctcatcgaaggcgatctcaatccggacaacagcgacgtggacaagctattcatccagctagttcagacctacaaccagctctttgaagagaaccctattaacgcgagcggggtggacgccaaagccatcctttccgccaggctctctaagagccggagactcgagaaccttatcgctcagttgcccggcgagaagaaaaacggccttttcggcaacctgattgccctgtccttgggcttgacgccgaacttcaagagcaacttcgacctagctgaggatgctaaactgcagctgtccaaggacacttatgatgacgaccttgacaacctcctggcccaaattggggatcagtacgcggacctcttcctcgctgccaagaacctctccgacgcgattctgttgtcggacatcctccgcgtgaacaccgagattactaaggccccactgtccgcgagcatgatcaaacgctacgacgagcaccatcaggatctaactctgctcaaggcgctcgtccgtcagcaactccctgagaagtacaaggagatcttcttcgaccagtccaagaacggctacgccggttacattgatggtggcgcgagccaggaggagttctacaagttcatcaagcccatcttagagaagatggacggcaccgaagagcttctcgtgaagctcaacagggaggacctcctaaggaagcagcgcacctttgacaacgggtctattcctcatcagatccatctcggcgagctccacgcgattttgagacgccaagaggacttctacccgttcttgaaggacaaccgggagaagatcgagaagatcctcacgttcaggatcccttattacgtcggccccctagctaggggtaactcgagattcgcgtggatgaccagaaagtccgaggaaaccattaccccgtggaactttgaagaggtggtcgacaagggcgcgagcgcgcaatcgttcattgagcggatgaccaacttcgacaagaacctaccgaacgagaaggtcctgccgaaacactccctgctctacgagtacttcaccgtttataacgagctgacgaaggtcaagtacgtgactgagggcatgcgtaaaccagcgttcctcagcggtgagcagaagaaggctatcgtggacctgctctttaagacgaaccgcaaggtgaccgtgaagcagctcaaggaggattatttcaagaagatcgagtgcttcgactcagtggagattagcggcgtggaggaccgtttcaatgcgagcttaggtacctaccacgacctcctgaagatcatcaaggacaaggacttcctcgacaacgaggaaaacgaggacatcctggaggacatcgtgctcaccctgaccctctttgaagacagggagatgatcgaggaaaggttgaagacctacgcgcacctattcgacgacaaggtcatgaaacagctcaagcggaggagatacactggctggggcagactttcgcggaagctcatcaatgggatcagggacaagcagtccggcaagaccatcctggacttcttgaagagcgacggcttcgctaatcgcaacttcatgcagctcattcatgacgactccctaacattcaaggaggacatccaaaaggcgcaagtctcgggtcagggggattctttgcacgagcacatcgcaaacctcgccggttctccggccattaaaaagggcatccttcagacggtcaaggtggtcgacgagctcgtgaaggttatgggccggcataagcccgagaacatagtgatcgagatggctagggagaaccagaccacgcaaaagggccagaagaactcacgtgagcgcatgaagaggatcgaggaaggtatcaaggagctgggctctcagattctcaaggagcatccggtcgagaacacccagcttcagaacgagaagctgtacctctactacctccagaatggccgcgacatgtacgttgatcaggagttagatattaatcggctgagcgactacgacgttgaccacatcgtgccccagtcgttcctcaaggatgactccatcgacaacaaggtcctgaccagatcggacaagaaccgtggtaagagcgacaatgtgccctccgaggaggtggtcaaaaagatgaagaactattggcggcagttgctgaacgccaagctcatcacccagcgaaagttcgataatttaacgaaggccgaacgagggggcctttccgaactcgataaggccggtttcatcaagcgtcagctggttgagacgcgacagatcacgaagcatgtggcccagattctggacagccggatgaacacgaagtacgacgagaacgacaaacttattcgggaggtgaaggtgataaccctcaagtccaagttagtgtcggacttccgcaaggactttcagttctataaagtgcgcgagatcaacaattaccaccacgcccacgacgcctacttgaatgccgtggtcggcacggcgctcatcaaaaagtaccccaagctggagagcgagttcgtctacggcgactacaaggtttatgacgtcaggaagatgattgcgaagtcagagcaagagatcgggaaggccaccgccaagtacttcttctactcgaacatcatgaacttctttaagaccgagatcaccctcgccaatggggagatccggaaacgtccccttatcgagacgaacggcgagactggcgagatagtctgggacaaaggtcgcgatttcgccaccgttcgaaaggtgctctcaatgcctcaggtcaatatcgtgaagaagaccgaggttcagacggggggcttcagcaaggagtccatcctccctaagcgcaactctgacaagctgatcgcccggaaaaaggattgggacccgaaaaagtacggcggcttcgattctcccaccgtagcttatagcgtgctcgtggttgccaaggtggagaagggcaagagcaagaaactgaagtcggtcaaggagttactcggcatcacaatcatggagcgcagctcgtttgaaaagaacccgatcgacttcctggaggccaagggctataaggaggtgaagaaggacctcatcattaagctgccgaagtacagcttgttcgagctcgagaatggcaggaagaggatgctagcttcggccggcgaacttcaaaaagggaacgagctcgcgttgccgtcgaagtacgtgaacttcctctacttagctagtcactatgaaaagctgaagggctcgccggaggacaacgagcagaagcagctcttcgtcgagcaacacaagcactacctcgacgagatcatcgagcagataagtgagttcagcaagcgcgtgatcctcgctgatgccaacctcgataaggtgctcagcgcctacaataagcaccgagacaagccgattcgggagcaggcggagaacatcattcatttatttacgctcactaacctaggcgctccggctgccttcaagtacttcgacaccacgatcgacaggaagcgctacaccagcaccaaagaagtcctcgacgcgacgctcattcaccagagcatcacagggctctacgagactcggatcgacttgtcgcaattgggtggggataagaggccggccgcgactaaaaaggccggccaagccaagaaaaagaaatga

SEQ ID NO: 6 - protein, 1401 aa, Artificial Sequence [sequence omitted]